(19) United States
(12) Patent Application Publication
(10) Pub. No.: US 2017/0258815 A1
(43) Pub. Date: Sep. 14, 2017

(54) CORTICOSTEROID FORMULATIONS AND METHODS FOR THE TREATMENT OF JOINT PAIN IN PATIENTS WITH DIABETES

(71) Applicant: Flexion Therapeutics, Inc., Burlington, MA (US)

(72) Inventor: Neil Bodick, Boston, MA (US)

(21) Appl. No.: 15/458,383

(22) Filed: Mar. 14, 2017

Related U.S. Application Data
(60) Provisional application No. 62/307,879, filed on Mar. 14, 2016.
(60) Provisional application No. 62/323,360, filed on Apr. 15, 2016.

(51) Int. Cl. A61K 31/58; A61K 9/00; A61K 9/16

(52) U.S. Cl. CPC A61K 31/58; A61K 9/1647; A61K 9/0019

(57) ABSTRACT

This invention relates to the use of corticosteroids in patients with diabetes, including patients with type 2 diabetes, to treat pain, including pain caused by inflammatory diseases such as osteoarthritis or rheumatoid arthritis without increasing or otherwise significantly impacting blood glucose concentrations in diabetic patients, and to slow, arrest or reverse structural damage to tissues caused by an inflammatory disease, for example damage to articular and/or periarticular tissues caused by osteoarthritis or rheumatoid arthritis without increasing or otherwise impacting blood glucose concentrations. More specifically, a formulation of triamcinolone acetonide (TCA) is administered locally to diabetes patients, including type 2 diabetes patients, as a sustained release dosage form (with or without an immediate release component) that results in efficacy levels accompanied by clinically insignificant or no measurable effect on blood glucose levels.

### RELATED APPLICATIONS

This application claims the benefit of U.S. Provisional Application No. 62/307,879, filed Mar. 14, 2016, and U.S. Provisional Application No. 62/323,360, filed Apr. 15, 2016, the contents of which are incorporated herein by reference in their entirety.

### FIELD OF THE INVENTION

This invention relates to the use of corticosteroids in patients with diabetes, including type 2 diabetes, to treat pain, including pain caused by inflammatory diseases such as osteoarthritis or rheumatoid arthritis without increasing or otherwise significantly impacting blood glucose concentrations in diabetic patients, and to slow, arrest or reverse structural damage to tissues caused by an inflammatory disease, for example damage to articular and/or peri-articular tissues caused by osteoarthritis or rheumatoid arthritis without increasing or otherwise impacting blood glucose concentrations. More specifically, a formulation of triamcinolone acetonide (TCA) is administered locally to diabetes patients, e.g., type 2 diabetes patients, as a sustained release dosage form (with or without an immediate release component) that results in efficacy levels accompanied by clinically insignificant or no measurable effect on blood glucose concentrations.

### BACKGROUND OF THE INVENTION

Corticosteroids influence all tissues of the body and produce various cellular effects. These steroids regulate carbohydrate, lipid, protein biosynthesis and metabolism, and water and electrolyte balance. Corticosteroids influencing cellular biosynthesis or metabolism are referred to as glucocorticoids while those affecting water and electrolyte balance are mineralocorticoids. Both glucocorticoids and mineralocorticoids are released from the cortex of the adrenal gland.

The administration of corticosteroids, particularly for extended periods of time, can have a number of unwanted side effects. The interdependent feedback mechanism between the hypothalamus, which is responsible for secretion of corticotrophin-releasing factor, the pituitary gland, which is responsible for secretion of adrenocorticotropic hormone, and the adrenal cortex, which secretes cortisol, is termed the hypothalamic-pituitary-adrenal (HPA) axis. The HPA axis may be suppressed by the administration of corticosteroids, leading to a variety of unwanted side effects.

Accordingly, there is a medical need to extend the local duration of action of corticosteroids, while reducing the systemic side effects associated with that administration. In addition, there is a medical need to slow, arrest, reverse or otherwise inhibit structural damage to tissues caused by inflammatory diseases such as damage to articular tissues resulting from various patient populations diagnosed with, predisposed to, or otherwise suffering from osteoarthritis or rheumatoid arthritis, including patients with other disorders such as diabetes, including type 2 diabetes.

### SUMMARY OF THE INVENTION

Disclosed herein are compositions and methods for the treatment of pain and inflammation in patients with diabetes using corticosteroids. In some embodiments, the compositions and methods provided herein are useful for the treatment of pain and inflammation in patients with type 2 diabetes. Type 2 diabetes, also known as diabetes mellitus type 2 (formerly noninsulin-dependent diabetes mellitus (NIDDM) or adult-onset diabetes) is a metabolic disorder that is characterized by high blood sugar in the context of insulin resistance and relative lack of insulin. There are subpopulations of patients with type 2 diabetes who also experience one or more symptoms of osteoarthritis (OA), with a prevalence of knee OA among these subpopulations. In some embodiments, the compositions and methods provided herein are useful for the treatment of pain and inflammation in patients with type 1 diabetes. Type 1 diabetes, also known as diabetes mellitus type 1 or T1D, (formerly insulin-dependent diabetes or juvenile diabetes) is a form of diabetes mellitus that results from the autoimmune destruction of the insulin-producing beta cells in the pancreas. The subsequent lack of insulin leads to increased glucose in blood and urine. There are subpopulations of patients with type 1 diabetes who also experience one or more symptoms of osteoarthritis (OA), with a prevalence of knee OA among these subpopulations.

The compositions and methods provided herein use one or more corticosteroids in a microparticle formulation in the treatment of patients with diabetes, including type 2 diabetes, without increasing or otherwise impacting blood glucose concentrations in patients. Typically, a patient's blood glucose concentration is elevated following administration, e.g., intra-articular administration, of a corticosteroid (see e.g., Habib G S, Miari, Wali, JCR Journal of Clinical Rheumatology, September 2011—Volume 17—Issue 6—pp 302-305). This elevation is typically seen within 72 hours post-administration and can last for upwards of a week or longer. In contrast, the compositions and methods provided herein use controlled-release microparticle formulations that provide continued, sustained release of one or more corticosteroids for a desired length of time. Thus, the controlled-release microparticle formulations provided herein reduce systemic exposure to the corticosteroid(s) and prolong synovial residence of the corticosteroid(s) as compared to current corticosteroid therapies, particularly to therapies that use immediate release formulations or other standard TCA crystalline suspensions.

Preferably, the corticosteroid is triamcinolone acetonide (TCA) and the microparticle is made from a poly(lactic-co-glycolic) acid copolymer (PLGA). These TCA containing PLGA microparticles and formulations thereof are collectively referred to herein as “TCA/PLGA microparticles” and “TCA/PLGA microparticle formulations,” where these terms are used interchangeably. The target for the TCA/PLGA microparticles and microparticle formulations described herein is about 22% to 28% triamcinolone acetonide, e.g., about 25% triamcinolone acetonide, in 75:25 PLGA with molecular weight of 50-54 kDa, inherent viscosity 0.40-0.46 dL/g, and the volumetric particle size of the microparticles is typically in the range of about 29-75 μm. TCA/PLGA microparticles and formulations described herein include one or more TCA/PLGA microparticles that have these specific properties, or a population of TCA/PLGA microparticles having an average of around 25% triamcinolone acetonide in 75:25 PLGA with molecular weight of 50-54 kDa, inherent viscosity 0.40-0.46 dL/g, and the volumetric particle size of the microparticles is typically in the range of about 29-75 μm.

In some embodiments, TCA/PLGA microparticle formulations are administered at a TCA dose in the range of about 10 mg to about 50 mg. In some embodiments, the TCA/PLGA microparticle formulations are administered at a TCA dose in the range of about 30 mg to about 50 mg. In some embodiments, the TCA/PLGA microparticle formulations are administered at a TCA dose in the range of about 31 mg to about 50 mg, about 32 mg to about 50 mg, about 33 mg to about 50 mg, about 34 mg to about 50 mg, about 35 mg to about 50 mg, about 36 mg to about 50 mg, about 37 mg to about 50 mg, about 38 mg to about 50 mg, about 39 mg to about 50 mg, about 40 mg to about 50 mg, about 41 mg to about 50 mg, about 42 mg to about 50 mg, about 43 mg to about 50 mg, about 44 mg to about 50 mg, about 45 mg to about 50 mg, about 46 mg to about 50 mg, about 47 mg to about 50 mg, about 48 mg to about 50 mg, or about 49 mg to about 50 mg. In some embodiments, the TCA/PLGA microparticle formulations are administered at a TCA dose in the range of about 30 mg to about 40 mg, about 31 mg to about 40 mg, about 32 mg to about 40 mg, about 33 mg to about 40 mg, about 34 mg to about 40 mg, about 35 mg to about 40 mg, about 36 mg to about 40 mg, about 37 mg to about 40 mg, about 38 mg to about 40 mg, or about 39 mg to about 40 mg. In some embodiments, the TCA/PLGA microparticle formulations are administered at a TCA dose of about 40 mg.

In some embodiments, the TCA/PLGA microparticles are administered in a formulation having a viscosity in the range of about 2.7 centipoise (cP) to about 3.5 cP. In some embodiments, the TCA/PLGA microparticles are administered in a formulation having a viscosity in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TCA/PLGA microparticle formulations are administered at a TCA dose in the range of about 10 mg to about 50 mg and in a formulation having a viscosity in the range of about 2.7 cP to about 3.5 cP. In some embodiments, TCA/PLGA microparticle formulations are administered at a TCA dose in the range of about 10 mg to about 50 mg and in a formulation having a viscosity of in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TCA/PLGA microparticle formulations are administered at a TCA dose in the range of about 10 mg to about 50 mg and in a formulation having a viscosity of about 3.0 cP.

In some embodiments, the TCA/PLGA microparticles are administered as a suspension having a viscosity in the range of about 2.7 centipoise (cP) to about 3.5 cP. In some embodiments, the TCA/PLGA microparticles are administered as a suspension having a viscosity in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TCA/PLGA microparticle formulations are administered at a TCA dose in the range of about 10 mg to about 50 mg and as a suspension having a viscosity in the range of about 2.7 cP to about 3.5 cP. In some embodiments, TCA/PLGA microparticle formulations are administered at a TCA dose in the range of about 10 mg to about 50 mg and as a suspension having a viscosity of in the range of about 2.8 cP to about 3.5 cP, about 2.9 cP to about 3.5 cP, about 3.0 cP to about 3.5 cP, about 3.1 cP to about 3.5 cP, about 3.2 cP to about 3.5 cP, about 3.3 cP to about 3.5 cP, about 3.4 cP to about 3.5 cP, about 2.8 cP to about 3.2 cP, about 2.9 cP to about 3.2 cP, about 3.0 cP to about 3.2 cP, about 2.8 cP to about 3.1 cP, about 2.9 cP to about 3.1 cP, about 3.0 cP to about 3.1 cP, about 2.8 cP to about 3.0 cP, or about 2.9 cP to about 3.0 cP. In some embodiments, TCA/PLGA microparticle formulations are administered at a TCA dose in the range of about 10 mg to about 50 mg and as a suspension having a viscosity of about 3.0 cP.

In some embodiments, the microparticles have a mean diameter in the range of 10-100 μm, for example, as detected by laser light scattering methods. In some embodiments, the microparticles have a mean diameter in the range of 20-100 μm, 20-90 μm, 30-100 μm, 30-90 μm, or 10-90 μm. It is understood that these ranges refer to the mean diameter of all microparticles in a given population. The diameter of any given individual microparticle could be within a standard deviation above or below the mean diameter.

The TCA/PLGA microparticle formulations provided herein are effective at treating pain and/or inflammation in patients with diabetes, including type 2 diabetes without increasing or otherwise impacting blood glucose concentrations in patients. The TCA/PLGA microparticle formulations provided herein are administered at a therapeutically effective concentration that treats, prevents, delays the progression of, or otherwise alleviates at least one symptom of osteoarthritis in a patient with diabetes, including type 2 diabetes, such that the therapeutically effective concentration results in efficacy levels that are not accompanied by clinically insignificant or no measurable effect on blood glucose concentrations and/or do not trigger or otherwise produce a significant elevation in the patient's blood glucose concentrations.

In some embodiments, a patient's blood glucose concentration following administration, e.g., following intra-articular administration, of the therapeutically effective concentration of the TCA/PLGA microparticle formulation is lower than the blood glucose concentration of a patient following administration, e.g., following intra-articular administration, of an immediate release TCA formulation or other standard triamcinolone acetonide (TA) crystalline suspension (“TAcs”). In some embodiments, a patient's blood glucose concentration following administration, e.g., following intra-articular administration, of the therapeutically effective concentration of the TCA/PLGA microparticle formulation is significantly lower than the blood glucose concentration of a patient following administration, e.g., following intra-articular administration, of an immediate release TCA formulation or other standard TA crystalline suspension (“TAcs”).

In some embodiments, a patient's blood glucose concentration following administration, e.g., following intra-articular administration, of the therapeutically effective concentration of the TCA/PLGA microparticle formulation is no more than 5-fold greater than the upper limit of a control blood glucose concentration. For example, a control blood glucose concentration is the blood glucose concentration of a normal, healthy patient (i.e., one who has not been diagnosed with diabetes). A normal fasting blood glucose concentration (i.e., no food for eight hours) is in the range of about 70 to 99 mg/dL, and a normal blood glucose concentration two hours after eating is less than 140 mg/dL.

In some embodiments, a patient's blood glucose concentration following administration, e.g., following intra-articular administration, of the therapeutically effective concentration of the TCA/PLGA microparticle formulation is between twofold and 5-fold greater than the upper limit of a control blood glucose concentration, for example, the blood glucose concentration of a normal, healthy patient. For example, the patient's blood glucose concentration following administration, e.g., following intra-articular administration, of the TCA/PLGA microparticle formulation is at least twofold, at least threefold, at least 4-fold, at least 5-fold or more higher than the upper limit of a control blood glucose concentration.

In some embodiments, a patient's blood glucose concentration following administration, e.g., following intra-articular administration, of the therapeutically effective concentration of the TCA/PLGA microparticle formulation is no more than 600 mg/dL. In some embodiments, a patient's blood glucose concentration following administration, e.g., following intra-articular administration, of the therapeutically effective concentration of the TCA/PLGA microparticle formulation is no more than 500 mg/dL. In some embodiments, a patient's blood glucose concentration following administration, e.g., following intra-articular administration, of the therapeutically effective concentration of the TCA/PLGA microparticle formulation is no more than 400 mg/dL. In some embodiments, a patient's blood glucose concentration following administration, e.g., following intra-articular administration, of the therapeutically effective concentration of the TCA/PLGA microparticle formulation is no more than 300 mg/dL. In some embodiments, a patient's blood glucose concentration following administration, e.g., following intra-articular administration, of the therapeutically effective concentration of the TCA/PLGA microparticle formulation is no more than 200 mg/dL. In some embodiments, a patient's blood glucose concentration following administration, e.g., following intra-articular administration, of the therapeutically effective concentration of the TCA/PLGA microparticle formulation is no more than 100 mg/dL.

The TCA/PLGA microparticle formulations provided herein are effective at treating pain and/or inflammation in patients with diabetes, including patients with type 2 diabetes, with minimal long-term side effects of corticosteroid administration, including for example, prolonged suppression of the HPA axis. The TCA/PLGA microparticle formulations are suitable for administration, for example, local administration by injection into a site at or near the site of a patient's pain and/or inflammation. The TCA/PLGA microparticle formulations provided herein are effective in slowing, arresting, reversing or otherwise inhibiting structural damage to tissues associated with progressive disease with minimal long-term side effects of TCA/PLGA administration, including for example, prolonged suppression of the HPA axis. The TCA/PLGA microparticle formulations are suitable for administration, for example, local administration by injection into a site at or near the site of structural tissue damage. As used herein, “prolonged” suppression of the HPA axis refers to levels of cortisol suppression greater than 40%, preferably greater than 35% by day 14 post-administration, for example post-injection. The TCA/PLGA microparticle formulations provided herein deliver the TCA in a dose and in a controlled or sustained release manner such that the levels of cortisol suppression are at or below 40%, preferably 35% by day 14 post-administration, for example post-injection. In some embodiments, the TCA/PLGA microparticle formulations provided herein deliver the TCA in a dose and in a controlled or sustained release manner such that the levels of cortisol suppression are negligible, clinically insignificant/inconsequential and/or undetectable by 14 post-administration, for example post-injection. In some embodiments, the TCA/PLGA microparticle formulations provided herein deliver the TCA in a dose and in a controlled or sustained release manner such that the levels of cortisol suppression are negligible at any time post-injection. Thus, the TCA/PLGA microparticle formulations in these embodiments are effective in the absence of any significant HPA axis suppression. Administration of the TCA/PLGA microparticle formulations provided herein can result in initial HPA axis suppression, for example, within the first few days, within the first two days and/or within the first 24 hours post-injection, but by day 14 post-injection, suppression of the HPA axis is less than 40%, preferably 35%.

In certain embodiments, a sustained release form of TCA/PLGA microparticles is administered locally to treat pain and inflammation in patients with diabetes, including type 2 diabetes. Local administration of a TCA/PLGA microparticle formulation can occur, for example, by injection into the intra-articular space or peri-articular space at or near the site of a patient's pain. In certain embodiments, the formulation additionally contains an immediate release component. In certain preferred embodiments of the invention, a sustained release form of TCA/PLGA microparticles is administered (e.g., by single injection or as sequential injections) into an intra-articular space for the treatment of pain, for example, due to osteoarthritis, rheumatoid arthritis, gouty arthritis and/or other joint disorders, or into local tissues affected by bursitis, tenosynovitis, epicondylitis, synovitis and/or other disorders. In certain preferred embodiments of the invention, a sustained release form of TCA/PLGA microparticles is administered (e.g., by single injection or as sequential injections) into an intra-articular space to slow, arrest, reverse or otherwise inhibit structural damage to tissues associated with progressive disease such as, for example, the damage to cartilage associated with progression of osteoarthritis. The TCA/PLGA microparticles described herein are also useful in the treatment of a systemic disorder for which TCA treatment would be required or otherwise therapeutically beneficial.

In one aspect, a TCA/PLGA microparticle formulation is provided the TCA/PLGA microparticle formulation provides at least two weeks, preferably at least three weeks, including up to and beyond 30 days, or 60 days, or 90 days of a sustained, steady state release of TCA. In one aspect, a TCA/PLGA microparticle formulation is provided wherein the TCA/PLGA microparticle formulation provides at least two weeks, preferably at least three weeks, including up to and beyond 30 days, or 60 days, or 90 days of a sustained, steady state release of TCA at a rate that does not adversely suppress the HPA axis. The duration of the release of TCA from the TCA/PLGA microparticles can vary in relation to the total number of TCA/PLGA microparticles contained in a given formulation. In the TCA/PLGA microparticle formulations described herein, when administered at a dose around 10 mg, the TCA/PLGA microparticles provide at least 6 weeks of a sustained, steady state release of TCA.

In some embodiments, the TCA/PLGA microparticle formulation retains sustained efficacy even after the TCA is no longer resident at the site of administration, for example, in the intra-articular space, and/or after the TCA is no longer detected in the systemic circulation. The TCA/PLGA microparticle formulation retains sustained efficacy even after the TCA/PLGA microparticle formulation is no longer resident at the site of administration, for example, in the intra-articular space, and/or the released TCA is no longer detected in the systemic circulation. The TCA/PLGA microparticle formulation retains sustained efficacy even after the TCA/PLGA microparticle formulation ceases to release therapeutically effective amounts of TCA. For example, in some embodiments, the TCA released by the TCA/PLGA microparticle formulation retains efficacy for at least one week, at least two weeks, at least three weeks, at least four weeks, at least five weeks, at least six weeks, at least seven weeks, at least eight weeks, at least nine weeks, at least twelve weeks, or more than twelve-weeks post-administration. In some embodiments, the TCA released by the TCA/PLGA microparticle formulation retains efficacy for a time period that is at least 1.1 times as long, at least 1.2 times as long, at least 1.3 times as long, at least 1.4 times as long, at least 1.5 times as long, at least 1.6 times as long, at least 1.7 times as long, at least 1.8 times as long, at least 1.9 times as long, twice as long, at least 2.5 times as long, at least three times as long, or more than three times as long as the residency period for the TCA and/or the TCA/PLGA microparticle formulation. In some embodiments, the sustained, steady state release of TCA will not adversely suppress the HPA axis.

In some embodiments, a controlled or sustained-release TCA/PLGA formulation is provided wherein formulation may or may not exhibit an initial rapid release in addition to the sustained, steady state release of TCA for a second length of time of at least two weeks, preferably at least three weeks, including up to and beyond 30 days, or 60 days, or 90 days. The initial rapid release can be, for example, an initial “burst” of release within 1 hour of administration the TCA/PLGA microparticle formulation. The initial rapid release can be within the first 24 hours post-administration. In some embodiments, the TCA/PLGA formulation is provided wherein formulation may or may not exhibit an initial rapid release within the first 24 hours post-administration. In some embodiments, the TCA/PLGA formulation is provided wherein formulation may or may not exhibit an initial “burst” of rapid release within 1 hour post-administration. It should be noted that when TCA levels are measured in vitro, an initial rapid release or burst of TCA release from the TCA/PLGA microparticle formulation can be seen, but this initial rapid release or burst may or may not be seen in vivo. In some embodiments, the TCA/PLGA formulations may or may not exhibit an initial rapid release, and the sustained, steady state release of TCA occurs at a rate that does not suppress the HPA axis at a level greater than 50% at day 14 post-administration. In some embodiments, the sustained, steady state release of TCA will not adversely suppress the HPA axis, for example, the level of HPA axis suppression at or less than 40%, preferably 35% by day 14 post-administration. In some embodiments, the sustained, steady state release of TCA does not significantly suppress the HPA axis, for example, the level of HPA axis suppression is negligible, clinically insignificant/inconsequential and/or undetectable by day 14 post-injection. In some embodiments, the sustained, steady state release of TCA does not significantly suppress the HPA axis, for example, the level of HPA axis suppression is negligible at all times post-injection. In some embodiments, the length of sustained release is between 21 days and 90 days. In some embodiments, the length of sustained release is between 21 days and 60 days. In some embodiments, the length of sustained release is between 14 days and 30 days. In some embodiments, the length of the initial “burst” release is between 0 and 1 hour post-administration. In some embodiments, the length of the initial release is between 0 and 24 hours.

The disclosure provides methods of treating pain or inflammation in a patient with diabetes, e.g., type 2 diabetes, by administering to the patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40.

In some embodiments, the PLGA copolymer has a molar ratio of lactic acid:glycolic acid of 75:25. In some embodiments, the 22% to 28% of TCA in the microparticles comprises a total TCA load dose between 10 to 50 mg. In some embodiments, the formulation comprises a TCA dose in of about 40 mg. In some embodiments, the PLGA has an inherent viscosity in the range of 0.3 to 0.5 dL/g. In some embodiments, the TCA is released for between 14 days and 90 days. In some embodiments, the formulation is administered as one or more injections. In some embodiments, the injection is one or more local injections at a site of pain. In some embodiments, the injection is one or more intra-articular or periarticular injections. In some embodiments, the patient has osteoarthritis, rheumatoid arthritis, acute gouty arthritis, and/or synovitis.

The disclosure provides methods of treating pain or inflammation in a patient with diabetes, e.g., type 2 diabetes by administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40, and wherein the formulation releases TCA for at least 14 days at a rate that does not adversely suppress the hypothalamic-pituitary-adrenal axis (HPA axis).

In some embodiments, the PLGA copolymer has a molar ratio of lactic acid:glycolic acid of 75:25. In some embodiments, the 22% to 28% of TCA in the microparticles comprises a total TCA load dose between 10 to 50 mg. In some embodiments, the formulation comprises a TCA dose in of about 40 mg. In some embodiments, the PLGA has an inherent viscosity in the range of 0.3 to 0.5 dL/g. In some embodiments, the TCA is released for between 14 days and 90 days. In some embodiments, the formulation is administered as one or more injections. In some embodiments, the injection is one or more local injections at a site of pain. In some embodiments, the injection is one or more intra-articular or periarticular injections. In some embodiments, the patient has osteoarthritis, rheumatoid arthritis, acute gouty arthritis, and/or synovitis.

The disclosure provides methods of slowing, arresting or reversing progressive structural tissue damage associated with chronic inflammatory disease in a patient with diabetes, e.g., type 2 diabetes by administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40.

In some embodiments, the PLGA copolymer has a molar ratio of lactic acid:glycolic acid of 75:25. In some embodiments, the 22% to 28% of TCA in the microparticles comprises a total TCA load dose between 10 to 50 mg. In some embodiments, the formulation comprises a TCA dose in of about 40 mg. In some embodiments, the PLGA has an inherent viscosity in the range of 0.3 to 0.5 dL/g. In some embodiments, the TCA is released for between 14 days and 90 days. In some embodiments, the formulation is administered as one or more injections. In some embodiments, the injection is one or more local injections at a site of pain. In some embodiments, the injection is one or more intra-articular or periarticular injections. In some embodiments, the patient has osteoarthritis, rheumatoid arthritis, acute gouty arthritis, and/or synovitis.

The disclosure provides methods of slowing, arresting or reversing progressive structural tissue damage associated with chronic inflammatory disease in a patient with diabetes, e.g., type 2 diabetes comprising administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40, wherein the formulation releases TCA for at least 14 days at a rate that does not adversely suppress the hypothalamic-pituitary-adrenal axis (HPA axis).

In some embodiments, the PLGA copolymer has a molar ratio of lactic acid:glycolic acid of 75:25. In some embodiments, the 22% to 28% of TCA in the microparticles comprises a total TCA load dose between 10 to 50 mg. In some embodiments, the formulation comprises a TCA dose in of about 40 mg. In some embodiments, the PLGA has an inherent viscosity in the range of 0.3 to 0.5 dL/g. In some embodiments, the TCA is released for between 14 days and 90 days. In some embodiments, the formulation is administered as one or more injections. In some embodiments, the injection is one or more local injections at a site of pain. In some embodiments, the injection is one or more intra-articular or periarticular injections. In some embodiments, the patient has osteoarthritis, rheumatoid arthritis, acute gouty arthritis, and/or synovitis.

The disclosure provides methods of slowing, arresting, reversing loss of or improving joint function in a patient with diabetes, e.g., type 2 diabetes, comprising administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa, and (ii) a lactic acid:glycolic acid molar ratio of 80:20 to 60:40.

In some embodiments, the PLGA copolymer has a molar ratio of lactic acid:glycolic acid of 75:25. In some embodiments, the 22% to 28% of TCA in the microparticles comprises a total TCA load dose between 10 to 50 mg. In some embodiments, the formulation comprises a TCA dose in of about 40 mg. In some embodiments, the PLGA has an inherent viscosity in the range of 0.3 to 0.5 dL/g. In some embodiments, the TCA is released for between 14 days and 90 days. In some embodiments, the formulation is administered as one or more injections. In some embodiments, the injection is one or more local injections at a site of pain. In some embodiments, the injection is one or more intra-articular or periarticular injections. In some embodiments, the patient has osteoarthritis, rheumatoid arthritis, acute gouty arthritis, and/or synovitis.

The TCA/PLGA microparticle formulations provided herein can be used in combination with any of a variety of therapeutics, also referred to herein as “co-therapies.”

For example, the TCA/PLGA microparticle formulations can be used in combination with an immediate release TCA (or other corticosteroid) solution or suspension or other standard triamcinolone acetonide (TA) crystalline suspension (“TAcs”), which provides high local exposures for between 1 day and 14 days following administration, e.g., intra-articular administration, and which produce systemic exposures that may be associated with transient suppression of the HPA axis. In some embodiments, the same corticosteroid, i.e., TCA, is used in both the TA crystalline suspension and sustained release components. In some embodiments, the TA crystalline suspension component contains a corticosteroid that is different from that of the sustained release component, i.e., the TA crystalline suspension component does not include TCA. In some embodiments, the sustained, steady state release of TCA will not adversely suppress the HPA axis. In some embodiments, the period of sustained release is between 21 days and 90 days. In some embodiments, the period of sustained release is between 21 days and 60 days. In some embodiments, the period of sustained release is between 14 days and 30 days. In some embodiments, the high local exposure attributable to the TA crystalline suspension component lasts for between 1 day and 14 days. In some embodiments, the high local exposure attributable to the TA crystalline suspension component lasts for between 1 day and 10 days. In some embodiments, the high local exposure attributable to the TA crystalline suspension component lasts between 1 days and 8 days. In some embodiments, the high local exposure attributable to the TA crystalline suspension component lasts between 1 days and 6 days. In some embodiments, the high local exposure attributable to the TA crystalline suspension component lasts for between 1 day and 4 days.

Suitable additional agents for use in combination with the TCA/PLGA microparticle formulations provided herein include hyaluronic acid preparations including but not limited to Synvisc One, Gel 200 and Supartz; NSAIDS including but not limited to aspirin, celecoxib (Celebrex), diclofenac (Voltaren), diflunisal (Dolobid), etodolac (Lodine), ibuprofen (Motrin), indomethacin, Indocin), ketoprofen (Orudis), ketorolac (Toradol), nabumetone (Relafen), naproxen (Aleve, Naprosyn), oxaprozin (Daypro), piroxicam (Feldene), salsalate (Amigesic), sulindac (Clinoril), tolmetin (Tolectin); biologics including but not limited to Actemra (tocilizumab), Enbrel (etanercept). Humira (adalimumab), Kineret (anakinra), Orencia (abatacept), Remicade (infliximab), Rituxan (rituximab), Cimzia (certolizumab), and Simponi (golimumab); disease modifying agents including but not limited to Methotrexate, Plaquenil (hydroxychloroquine) and Azulfidine (sulfasalazine), Minocin (minocycline); and other analgesic and anti-inflammatory agents including but not limited to p38 inhibitors JAC inhibitors, opioids, other corticosteroids, lidocaine, bupivacaine, ropivacaine, botulinum toxin A.

In some embodiments, the TCA/PLGA microparticle formulation and additional agent are formulated into a single therapeutic composition, and the TCA/PLGA microparticle formulation and additional agent are administered simultaneously. Alternatively, the TCA/PLGA microparticle formulation and additional agent are separate from each other, e.g., each is formulated into a separate therapeutic composition, and the TCA/PLGA microparticle formulation and the additional agent are administered simultaneously, or the TCA/PLGA microparticle formulation and the additional agent are administered at different times during a treatment regimen. For example, the TCA/PLGA microparticle formulation is administered prior to the administration of the additional agent, the TCA/PLGA microparticle formulation is administered subsequent to the administration of the additional agent, or the TCA/PLGA microparticle formulation and the additional agent are administered in an alternating fashion. As described herein, the TCA/PLGA microparticle formulation and additional agent are administered in single doses or in multiple doses.

In some embodiments, the TCA/PLGA microparticle formulation and the additional agent are administered by the same route. In some embodiments, the TCA/PLGA microparticle formulation and the additional agent are administered via different routes.

In some embodiments, the methods include the initial step of identifying a selected patient population for treatment with a TCA/PLGA microparticle formulation of the disclosure. In some embodiments, the patient is identified based on diagnosis of diabetes, e.g., type 2 diabetes. The World Health Organization defines type 2 diabetes by detection of a fasting plasma glucose level of greater than or equal to 7.0 mmol/l (126 mg/dl) or through the use of a glucose tolerance test, when two hours after the oral dose, the plasma glucose level is greater than or equal to 11.1 mmol/l (200 mg/dl).

In some embodiments, the diabetes patient population, e.g., type 2 diabetes patient population, is further identified or otherwise stratified based on pain level. On the 10 point Numerical Rating scale where 0 is no pain and 10 is the worst pain imaginable, patients with a score of 5 or above are an identified patient population. In some embodiments, the diabetes patient population, e.g., type 2 diabetes patient population, is further identified or otherwise stratified based on level joint movement. In some embodiments, the type 2 diabetes patient population is further identified or otherwise stratified based on level of joint deterioration. Patients with Osteoarthritis with scores of 2 and 3 in the Kellgren & Lawrence system for classification of knee radiographs are an identified population. In some embodiments, the diabetes patient population, e.g., the type 2 diabetes patient population, is further identified or otherwise stratified based on the occurrence of prior joint injury leading to deterioration of joint tissues.

It is contemplated that whenever appropriate, any embodiment of the present invention can be combined with one or more other embodiments of the present invention, even though the embodiments are described under different aspects of the present invention.

### BRIEF DESCRIPTION OF THE DRAWINGS

FIG. 1 is a graph depicting mean average blood glucose by hour (−72 hours to 72 hours).

FIG. 2 is a graph depicting mean change from baseline for average blood glucose (−72 hours to 72 hours).

FIG. 3 is a graph depicting mean change from baseline for average blood glucose (−48 hours to 48 hours).

FIG. 4 is a graph depicting mean average glucose by day.

FIG. 5 is a graph depicting average daily blood glucose by category (Day 1-2).

### DETAILED DESCRIPTION OF THE INVENTION

The disclosure provides compositions and methods for the treatment of pain and inflammation in patients with diabetes, including type 2 diabetes, using corticosteroids without increasing or otherwise impacting blood glucose concentrations in patients. Type 2 diabetes, also known as diabetes mellitus type 2 (formerly noninsulin-dependent diabetes mellitus (NIDDM) or adult-onset diabetes) is a metabolic disorder that is characterized by high blood sugar in the context of insulin resistance and relative lack of insulin. There are subpopulations of patients with type 2 diabetes who also experience one or more symptoms of osteoarthritis (OA), with a prevalence of knee OA among these subpopulations. In some embodiments, the compositions and methods provided herein are useful for the treatment of pain and inflammation in patients with type 1 diabetes. Type 1 diabetes, also known as diabetes mellitus type 1 or T1D, (formerly insulin-dependent diabetes or juvenile diabetes) is a form of diabetes mellitus that results from the autoimmune destruction of the insulin-producing beta cells in the pancreas. The subsequent lack of insulin leads to increased glucose in blood and urine. There are subpopulations of patients with type 2 diabetes who also experience one or more symptoms of osteoarthritis (OA), with a prevalence of knee OA among these subpopulations.

The compositions and methods provided herein use TCA in a PLGA microparticle formulation for use in treating patients with diabetes, including type 2 diabetes, without increasing or otherwise impacting blood glucose concentrations in patients. The TCA/PLGA microparticle formulations provided herein are administered at a therapeutically effective concentration that treats, prevents, delays the progression of, or otherwise alleviates at least one symptom of osteoarthritis in a patient with diabetes, including type 2 diabetes, such that the therapeutically effective concentration is accompanied by clinically insignificant or no measurable effect on blood glucose and/or does not trigger or otherwise produce a clinically significant elevation in the patient's blood glucose concentrations.

The TCA/PLGA microparticle formulations provided herein are effective at treating pain and/or inflammation with minimal prolonged suppression of the HPA axis and/or other long term side effects of TCA administration. The TCA/PLGA microparticle formulations provided herein are effective in slowing, arresting, reversing or otherwise inhibiting structural damage to tissues associated with progressive disease with minimal prolonged suppression of the HPA axis and/or other long term side effects of TCA administration. The TCA/PLGA microparticle formulations provided herein deliver TCA in a dose and in a sustained release manner such that the levels of cortisol suppression are at or below 40%, preferably 35% by day 14 post-injection. In some embodiments, the TCA/PLGA microparticle formulations provided herein deliver TCA in a dose and in a controlled or sustained release manner such that the levels of cortisol suppression are negligible, clinically insignificant/inconsequential and/or undetectable by 14 post-injection. Thus, the TCA/PLGA microparticle formulations in these embodiments are effective in the absence of any significant HPA axis suppression. Administration of the TCA/PLGA microparticle formulations provided herein can result in initial HPA axis suppression, for example, within the first few days, within the first two days and/or within the first 24 hours post-injection, but by day 14 post-injection, suppression of the HPA axis is less than 40%, preferably 35%.

The compositions and methods disclosed herein are useful for the treatment of osteoarthritis (OA) in patients with diabetes, including type 2 diabetes, without increasing or otherwise impacting blood glucose concentrations in patients. OA is a painful and debilitating musculoskeletal disease that is characterized by intra-articular (IA) inflammation, deterioration of articular cartilage, and degenerative changes to peri-articular and subchondral bone. (Creamer P, Hochberg M C. Osteoarthritis. Lancet 1997; 350(906):503-508; and Goldring S R, Goldring M B. Clinical aspects, pathology and pathophysiology of osteoarthritis. J Musculoskelet Neuronal Interact 2006; 6(4):376-378). Arthritis is the most common cause of disability in the United States (US) and OA is the most common joint disease, affecting 27 million Americans, with numbers expected to grow as a result of aging, obesity and sports injuries. Recent data suggest that OA accounts for over $185 billion of annual healthcare expenditures in the US, which does not include loss of productivity costs. We estimate that by 2030, 45 million people will have OA. OA commonly affect large weight-bearing joints like the knees and hips, but also occurs in the shoulders, hands, feet and spine. Patients with OA suffer from joint pain, tenderness, stiffness and limited movement. As the disease progresses, it becomes increasingly painful and debilitating, culminating, in many cases, in the need for total joint arthroplasty.

Current Guidelines from the American College of Rheumatology (ACR), Osteoarthritis Research Society International (OARSI) and the European League against Rheumatism (EULAR) recommend the use of IA corticosteroids for short-term acute pain relief (ACR Subcommittee 2000; Hochberg M C, Altman R D, April K T, Benkhalti M, Guyatt G, McGowan J, Towheed T, Welch V, Wells G, Tugwell P. American College of Rheumatology 2012 Recommendations for the Use of Nonpharmacologic and Pharmacologic Therapies in Osteoarthritis of the Hand, Hip, and Knee. Arthritis Care & Research 2012; 64:465-474).

While historically OA has been considered a non-inflammatory disease, it is increasingly being recognized that chronic synovitis occurs in all stages of knee OA. (Benito M J, Veale D J, FitzGerald O, van den Berg W B, Bresnihan B. Synovial tissue inflammation in early and late osteoarthritis. Ann Rheum Dis 2005; 64:1263-1267; Sellam J and Berenbaum F. The role of synovitis in pathophysiology and clinical symptoms of osteoarthritis. Nature Reviews Rheumatology 2010; 6:625-635; and Wenham C Y J and Conaghan P G. The role of synovitis in osteoarthritis. Ther Adv Musculoskel Dis 2010; 2:349-359). As synovial inflammation is correlated with clinical symptoms and joint degeneration, it should be an important target for therapeutic intervention. The inflamed synovium may well be the target for IA corticosteroids which are widely used in knee OA. (Ayral X, Pickering E H, Woodworth T G, Mackillop N, Dougados M. Synovitis: a potential predictive factor of structural progression of medial tibiofemoral knee osteoarthritis—results of a 1 year longitudinal arthroscopic study in 422 patients. Osteoarthritis Cartilage 2005; 13:361-7).

Approximately 14.4% of patients with OA have been diagnosed with diabetes mellitus (DM) (Louati et al, Association between diabetes mellitus and osteoarthritis: systematic literature review and meta-analysis. RMD Open. 2015; 1(1):e000077). In this population, IA corticosteroids can increase blood glucose (BG) for approximately 72 hours post-injection (Habib et al, Increased blood glucose levels following intra-articular injection of methylprednisolone acetate in controlled diabetic patients with symptomatic osteoarthritis of the knee. Ann Rheum Dis 2008; 67:1790-1791).

IA corticosteroids are generally considered safe with a low incidence of local or systemic adverse effects. (Bellamy N, Campbell J, Robinson V, Gee T, Bourne R, Wells G. Intraarticular corticosteroid for treatment of osteoarthritis of the knee. Cochrane Database Syst Rev 2006; Issue 2. Art No.: CD005328; and Jüni, P Roman Hari Anne W S Rutjes, Roland Fischer, Maria G Silletta, Stephan Reichenbach, Bruno R da Costa, Intra-articular corticosteroid for knee osteoarthritis Editorial Group: Cochrane Musculoskeletal Group Published Online: 22 Oct. 2015, Assessed as up-to-date: 3 Feb. 2015, DOI: 10.1002/14651858.CD005328). However, transient increases in blood glucose levels even among controlled diabetics are a known side effect of currently available IA corticosteroids. (Habib G S. Systemic effects of intra-articular corticosteroids. Clin Rheumatol 2009; 28:749-756). In a controlled study comparing IA injection of TCA or triamcinolone hexacetonide (TAH) to IA hyaluronic acid, both corticosteroid preparations resulted in significantly increased levels of blood glucose in patients with controlled DM and OA of the knee. (Habib G S, Miari, Wali, JCR Journal of Clinical Rheumatology, September 2011—Volume 17—Issue 6—pp 302-305). The studies provided herein demonstrate that the TCA/PLGA microparticles of the disclosure administered locally to a patient at therapeutically efficacious levels are accompanied by clinically insignificant or no measurable effect on blood glucose and/or do not result in significantly increased blood glucose concentrations in patients following administration, e.g., following intra-articular administration.

Type 2 diabetes is estimated to affect 26 million persons in the United States alone, and the prevalence of type 2 diabetes in the United States is expected to triple by 2050. (Centers for Disease Control and Prevention, National diabetes fact sheet: national estimates and general information on diabetes and prediabetes in the United States, 2011. Atlanta: Department of Health and Human Services, Centers for Disease Control and Prevention, 2011; available on the CDC website). Among patients with type 1 diabetes and type 2 diabetes the overall prevalence of OA approaches 30.0%, with a prevalence of knee OA among these patients of 17.2%. (Louati K, Vidal C, Berenbaum F, et al. Association between diabetes mellitus and osteoarthritis: systematic literature review and meta-analysis. RMD Open 2015; 1:e000077. doi: 10.1136/rmdopen-2015-000077). The elucidation of shared mechanisms that contribute to the development of both type 2 diabetes and OA which are increasingly viewed as inflammatory disorders is an area of active investigation. In a recent study Type 2 diabetes was shown to predict joint space narrowing among males with symptomatic OA. (Eymard F, Parsons C, Edwards M H, et al. Diabetes is a risk factor for knee osteoarthritis progression. Osteoarthritis Cartilage 2015. Published Online First: 3 Feb. 2015). Furthermore, it has been hypothesized that hyperglycemia may contribute to the progression of OA through the generation of advanced glycation end products (AGEs), oxidative stress and the induction of inflammatory mediators. (Berenbaum F. Diabetes-induced osteoarthritis: from a new paradigm to a new phenotype. Ann Rheum Dis 2011; 70:1354-6).

Immediate release IA corticosteroids such as standard TA crystalline suspension (TAcs) rapidly diffuse from the joint space and enter the systemic circulation (Derendorf H, Mollmann H, Grüner A, Haack D, Gyselby G. Pharmacokinetics and pharmacodynamics of glucocorticoid suspensions after intra-articular administration. Clin Pharmacol Ther 1986; 39:313-31). However, consistent with slow release of TCA from PLGA microspheres in the synovial tissues, TCA/PLGA microparticles substantially reduce systemic exposure to TCA and prolong synovial residence of TCA relative to 40 mg TAcs. In a previous pharmacokinetic study in patients with knee osteoarthritis, TCA/PLGA microparticles was associated with slow absorption into the systemic circulation with a peak plasma concentration of 0.88 ng/ml occurring in the first 4 hours post-injection. (Bodick N, Lufkin J, Willwerth C, Blanks R, Inderjeeth C, Kumar A, Clayman M., TCA/PLGA microparticles prolong the residency of triamcinolone acetonide in the synovial tissues of patients with knee osteoarthritis. Osteoarthritis Cartilage. 2013; 21:(Suppl); S144-5). In contrast, the plasma profile of TAcs was characterized by rapid absorption of TCA into the systemic circulation with relatively high peak plasma levels (17.54 ng/ml) occurring in the first 4 hours post-injection. During weeks 1-6, 40 mg TCA/PLGA microparticles maintains a low plateau of systemic exposure, and the overall systemic AUC of TCA/PLGA microparticles is approximately one half that associated with 40 mg TAcs.

Previous studies have shown that the mean Cₘₐₓ for TCA in plasma following injection of the 40 mg dose of the TCA/PLGA microparticles of the disclosure was 20-fold less than that produced by the matching 40 mg dose of TAcs. The absence of this early peak accounts for the substantial reduction in % Fluctuation with TCA/PLGA microparticles relative to TAcs, as all doses of TCA/PLGA microparticles maintain relatively constant plateau concentrations of TCA in plasma over 12 weeks.

The TCA/PLGA microparticle formulations provided herein are an extended-release formulation of triamcinolone acetonide (TCA) for IA administration that is being developed for the treatment of patients with OA of the knee. These formulations are described, for example, in PCT Publication WO 2012/019009, the contents of which are hereby incorporated by reference in their entirety.

Available clinical and nonclinical data indicate that TCA/PLGA microparticles is safe and well-tolerated and provides pain relief that is meaningfully better and more persistent than that provided by other standard triamcinolone acetonide (TA) crystalline suspension (“TAcs”). The safety and efficacy of these TCA/PLGA microparticle formulations is describe, for example, in Bodick N, Lufkin J, Willwerth C, Kumar A, Bolognese J, Schoonmaker C, Ballal R, Hunter D, Clayman, M. An Intra-Articular, Extended-Release Formulation of Triamcinolone Acetonide Prolongs and Amplifies Analgesic Effect in Patients with Osteoarthritis of the Knee, A Randomized Clinical Trial. J Bone Joint Surg Am. 2015; 97:877-88; and in Bodick N, Lufkin J, Willwerth C, Blanks R, Inderjeeth C, Kumar A, Clayman M., TCA/PLGA microparticles prolong the residency of triamcinolone acetonide in the synovial tissues of patients with knee osteoarthritis. Osteoarthritis Cartilage. 2013; 21:(Suppl), S144-5, the contents of each of which are hereby incorporated by reference in their entirety.

The studies presented herein were designed to evaluate the use of TCA/PLGA microparticles in patients with OA of the knee and type 2 diabetes to confirm that the pharmacokinetic profile of TCA/PLGA microparticles characterized by reduced systemic exposure and prolonged residency of TCA in the synovial tissues which results from the slow release of TCA from PLGA microspheres minimizes or eliminates the hyperglycemia that is characteristic of standard corticosteroid preparations administered by IA injection.

The studies provided herein were designed to assess the effects of a single intra-articular (IA) injection of 40 mg of TCA/PLGA microparticles, an extended release formulation of triamcinolone acetonide (TCA), on blood glucose concentrations in patients with type 2 diabetes, relative to 40 mg of TAcs. In addition, these studies were designed to assess the safety and general tolerability of a single IA injection of 40 mg of TCA/PLGA microparticles relative to 40 mg of TAcs.

The details of one or more embodiments of the invention are set forth in the accompanying description below. Although any methods and materials similar or equivalent to those described herein can be used in the practice or testing of the present invention, the methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present Specification will control.

“Patient” refers to a human diagnosed with a disease or condition that can be treated in accordance to the inventions described herein. In some embodiments it is contemplated that the formulations described herein may also be used in horses and other animals.

“Diagnosis” of a patient with osteoarthritis (OA) is accomplished using knee OA assessments according to the American College of Rheumatology Criteria for Classification of Idiopathic OA of the Knee (see e.g., Altman et al, “Development of criteria for the classification and reporting of osteoarthritis. Classification of osteoarthritis of the knee. Diagnostic and Therapeutic Criteria Committee of the American Rheumatism Association.” Arthritis Rheum., vol. 29(8): 1039-49 (1986)). Severity of diagnosed knee OA is assessed using the Kellgren-Lawrence Grade of Knee X-ray analysis. Briefly, the following scores are used in the Kellgren-Lawrence Grade of Knee X-ray:
Grade 0: no findings
Grade 1: doubtful narrowing of joint pace and possible osteophytic lipping
Grade 2: definite osteophytes and possible narrowing of joint space
Grade 3: moderate multiple osteophytes, definite narrowing of joint space and some sclerosis and possible deformity of bone ends
Grade 4: large osteophytes, marked narrowing of joint space, severe sclerosis and definite deformity of bone ends

“Adverse events” (AE) is any untoward medical occurrence in a patient or clinical investigation subject administered a pharmaceutical product and that does not necessarily have a causal relationship with this treatment. An AE can be, by way of non-limiting example, any unfavorable and unintended sign (including an abnormal laboratory finding), symptom, or disease temporally associated with the use of a medicinal (investigational) product, whether or not related to the medicinal (investigational) product; any clinically significant abnormality found on an ECG, laboratory test or physical examination; and/or any worsening (i.e., any clinical significant adverse change in frequency and/or intensity) of a preexisting condition, which is temporally associated with the use of the medicinal (investigational) product, is also an AE. A “serious adverse event” (SAE) is any untoward medical occurrence that, at any dose, results in death, is life-threatening, otherwise requires inpatient hospitalization or prolongation of existing hospitalization, results in permanent or significant disability/incapacity, and/or is a congenital anomaly/birth defect. Severity of AEs can be graded by the Principal Investigator using the Common Terminology Criteria for AEs (CTCAE) version 4.0. For AEs not listed in the CTCAE, the following definitions should be used:

| | |
|---|---|
| Grade 1 | Mild |
| | Symptomatic or mild symptoms |
| | Clinical or diagnostic observations only |
| | Intervention not indicated |
| Grade 2 | Moderate |
| | Minimal, local or noninvasive intervention indicated |
| | Limiting age-appropriate instrumental activities of daily |
| | living (ADL). Instrumental ADL refers to preparing meals, |
| | shopping for groceries or clothes, using the telephone, |
| | managing money, etc. |
| Grade 3 | Severe or medically significant but not immediately life- |
| | threatening |
| | Hospitalization or prolongation of hospitalization indicated |
| | Disabling |
| | Limiting self-care ADL. Self-care ADL refer to bathing, |
| | dressing and undressing, feeding self, using the toilet, taking |
| | medications, and not bedridden. |
| Grade 4 | Life-threatening consequences |
| | Urgent intervention indicated |
| Grade 5 | Death related to AE |

“Delivery” refers to any means used to place the drug into a patient. Such means may include without limitation, placing matrices into a patient that release the drug into a target area. One of ordinary skill in the art recognizes that the matrices may be delivered by a wide variety of methods, e.g., injection by a syringe, placement into a drill site, catheter or canula assembly, or forceful injection by a gun type apparatus or by placement into a surgical site in a patient during surgery.

The terms “treatment” and “treating” a patient refer to reducing, alleviating, stopping, blocking, delaying the progression, or preventing the symptoms of pain and/or inflammation in a patient. As used herein, “treatment” and “treating” includes partial alleviation of symptoms as well as complete alleviation of the symptoms for a time period. The time period can be hours, days, months, or even years.

By an “effective” amount or a “therapeutically effective amount” of a drug or pharmacologically active agent is meant a nontoxic but sufficient amount of the drug or agent to provide the desired effect, e.g., analgesia. An appropriate “effective” amount in any individual case may be determined by one of ordinary skill in the art using routine experimentation.

“Site of a patient's pain” refers to any area within a body causing pain, e.g., a knee joint with osteoarthritis, nerve root causing sciatic pain, nerve fibers growing into annular tears in discs causing back pain, temporomandibular joint (TMJ) pain, for example TMJ pain associated with temporomandibular joint disorder (TMD) or pain radiating from epidural or perineural spaces. The pain perceived by the patient may result from inflammatory responses, mechanical stimuli, chemical stimuli, thermal stimuli, as well as allodynia.

Additionally, the site of a patient's pain can comprise one or multiple sites in the spine, such as between the cervical, thoracic, or lumbar vertebrae, or can comprise one or multiple sites located within the immediate area of inflamed or injured joints such as the shoulder, hip, or other joints.

A “biocompatible” material refers to a material that is not toxic to the human body, it is not carcinogenic and it should induce limited or no inflammation in body tissues. A “biodegradable” material refers to a material that is degraded by bodily processes (e.g., enzymatic) to products readily disposable by the body or absorbed into body tissue. The biodegraded products should also be biocompatible with the body. In the context of intra-articular drug delivery systems for TCA and other corticosteroids, such polymers may be used to fabricate, without limitation: microparticles, micro-spheres, matrices, microparticle matrices, micro-sphere matrices, capsules, hydrogels, rods, wafers, pills, liposomes, fibers, pellets, or other appropriate pharmaceutical delivery compositions that a physician can administer into the joint. The biodegradable polymers degrade into non-toxic residues that the body easily removes or break down or dissolve slowly and are cleared from the body intact. The polymers may be cured ex-vivo forming a solid matrix that incorporates the drug for controlled release to an inflammatory region. Suitable biodegradable polymers may include, without limitation natural or synthetic biocompatible biodegradable material.

Descriptions of various embodiments of the invention are given below. Although these embodiments are exemplified with reference to treat joint pain associated with osteoarthritis, rheumatoid arthritis and other joint disorders, it should not be inferred that the invention is only for these uses. Rather, it is contemplated that embodiments of the present invention will be useful for treating other forms of joint pain by administration into articular and periarticular spaces. In addition, it will be understood that for some embodiments injection near a joint may be equivalent to injections in that joint. Any and all uses of specific words and references are simply to detail different embodiments of the present invention.

Local administration of a TCA/PLGA microparticle formulation can occur, for example, by injection into the intra-articular space or peri-articular space at or near the site of a patient's pain and/or structural tissue damage. Local injection of the formulations described herein into articular or periarticular spaces may be useful in the treatment of, for example, juvenile rheumatoid arthritis, sciatica and other forms of radicular pain (e.g., arm, neck, lumbar, thorax), psoriatic arthritis, acute gouty arthritis, Morton's neuroma, acute and subacute bursitis, acute and subacute nonspecific tenosynovitis and epicondylitis, and ankylosing spondylitis.

In one embodiment, the TCA/PLGA microparticle formulations provided herein are useful in treating, alleviating a symptom of, ameliorating and/or delaying the progression of sciatica. In one embodiment, TCA/PLGA microparticle formulations provided herein are useful in treating, alleviating a symptom of, ameliorating and/or delaying the progression of temporomandibular joint disorder (TMD).

Administration of a TCA/PLGA microparticle formulation to a patient suffering from an inflammatory disease such as osteoarthritis or rheumatoid arthritis, is considered successful if any of a variety of laboratory or clinical results is achieved. For example, administration of a TCA/PLGA microparticle formulation is considered successful if one or more of the symptoms associated with the disease is alleviated, reduced, inhibited or does not progress to a further, i.e., worse, state. Administration of a TCA/PLGA microparticle formulation is considered successful if the disease, e.g., an arthritic or other inflammatory disease, or any symptom thereof enters remission or does not progress to a further, i.e., worse, state.

Also, any and all alterations and further modifications of the invention, as would occur to one of ordinary skill in the art, are intended to be within the scope of the invention.

### TCA and Drug Dosage

Triamcinolone acetonide (TCA) associated with embodiments of the present invention has the following basic structure:

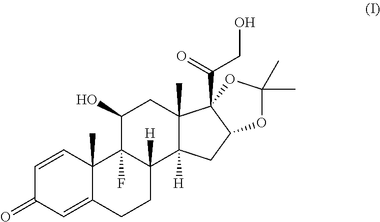

For the present invention non-limiting examples of TCA may include triamcinolone acetonide and/or pharmaceutically acceptable salts thereof.

Embodiments of the invention include using sustained release TCA/PLGA microparticles delivered to treat pain at dosages that do not adversely suppress the HPA axis. Such amounts delivered locally to relieve pain due to inflammation, will provide a systemic concentration that does not have a measurable adverse effect on the HPA axis (differences if any are not significant because any such differences are within normal assay variability) or, as desired, may have a measurable but clinically insignificant effect on the HPA axis (basal cortisol is suppressed to some measurable extent but stress responses are adequately preserved). Further embodiments of the invention may include doses during a second period of time selected to adjust for a change in sensitivity of the HPA axis to suppression following exposure during a first period of time to TCA.

In one preferred embodiment, a single component TCA/PLGA microparticle sustained release formulation releases a TCA dose (in mg/day) that suppresses the HPA axis by no more than between 5-40% at steady state, more preferably no more than between 10-35% at steady state. These doses are therapeutically effective without adverse side effects.

In another preferred embodiment, a single component TCA/PLGA microparticle sustained release formulation releases a dose (in mg/day) that does not measurably suppress the HPA axis at steady state. These doses are therapeutically effective without adverse side effects.

### Sustained Release Delivery Platforms

The manufacture of PLGA microparticles or methods of making biodegradable polymer microparticles are known in the art. PLGA microparticles are commercially available from a number of sources and/or can be made by, but not limited to, spray drying, solvent evaporation, phase separation, fluidized bed coating or combinations thereof.

If not purchased from a supplier, then the biodegradable PLGA copolymers may be prepared by the procedure set forth in U.S. Pat. No. 4,293,539 (Ludwig, et al.), the disclosure of which is hereby incorporated by reference in its entirety. Ludwig prepares such copolymers by condensation of lactic acid and glycolic acid in the presence of a readily removable polymerization catalyst (e.g., a strong acid ion-exchange resin such as Dowex HCR-W2-H). However, any suitable method known in the art of making the polymer can be used.

In the coacervation process, a suitable biodegradable polymer is dissolved in an organic solvent. Suitable organic solvents for the polymeric materials include, but are not limited to acetone, halogenated hydrocarbons such as chloroform and methylene chloride, aromatic hydrocarbons such as toluene, halogenated aromatic hydrocarbons such as chlorobenzene, and cyclic ethers such as dioxane. The organic solvent containing a suitable biodegradable polymer is then mixed with a non-solvent such as silicone based solvent. By mixing the miscible non-solvent in the organic solvent, the polymer precipitates out of solution in the form of liquid droplets. The liquid droplets are then mixed with another non-solvent, such as heptane or petroleum ether, to form the hardened microparticles. The microparticles are then collected and dried. Process parameters such as solvent and non-solvent selections, polymer/solvent ratio, temperatures, stirring speed and drying cycles are adjusted to achieve the desired particle size, surface smoothness, and narrow particle size distribution.

In the phase separation or phase inversion procedures entrap dispersed agents in the polymer to prepare microparticles. Phase separation is similar to coacervation of a biodegradable polymer. By addition of a nonsolvent such as petroleum ether, to the organic solvent containing a suitable biodegradable polymer, the polymer is precipitates from the organic solvent to form microparticles.

In the salting out process, a suitable biodegradable polymer is dissolved in an aqueous miscible organic solvent. Suitable water miscible organic solvents for the polymeric materials include, but are not limited to acetone, as acetone, acetonitrile, and tetrahydrofuran. The water miscible organic solvent containing a suitable biodegradable polymer is then mixed with an aqueous solution containing salt. Suitable salts include, but are not limited to electrolytes such as magnesium chloride, calcium chloride, or magnesium acetate and non-electrolytes such as sucrose. The polymer precipitates from the organic solvent to form microparticles, which are collected and dried. Process parameters such as solvent and salt selection, polymer/solvent ratio, temperatures, stirring speed and drying cycles are adjusted to achieve the desired particle size, surface smoothness, and narrow particle size distribution.

Alternatively, the microparticles may be prepared by the process of Ramstack et al., 1995, described in published international patent application WO 95/13799, the disclosure of which is incorporated herein in its entirety. The Ramstack et al. process essentially provides for a first phase, including an active agent and a polymer, and a second phase, that are pumped through a static mixer into a quench liquid to form microparticles containing the active agent. The first and second phases can optionally be substantially immiscible and the second phase is preferably free from solvents for the polymer and the active agent and includes an aqueous solution of an emulsifier.

In the spray drying process, a suitable biodegradable polymer is dissolved in a suitable solvent and then sprayed through nozzles into a drying environment provided with sufficient elevated temperature and/or flowing air to effectively extract the solvent.

Alternatively, a suitable biodegradable polymer can be dissolved or dispersed in supercritical fluid, such as carbon dioxide. The polymer is either dissolved in a suitable organic solvent, such as methylene chloride, prior to mixing in a suitable supercritical fluid or directly mixed in the supercritical fluid and then sprayed through a nozzle. Process parameters such as spray rate, nozzle diameter, polymer/solvent ratio, and temperatures, are adjusted to achieve the desired particle size, surface smoothness, and narrow particle size distribution.

In a fluidized bed coating, the drug is dissolved in an organic solvent along with the polymer. The solution is then processed, e.g., through a Wurster air suspension coating apparatus to form the final microcapsule product.

The microparticles can be prepared in a size distribution range suitable for local infiltration or injection. The diameter and shape of the microparticles can be manipulated to modify the release characteristics. In addition, other particle shapes, such as, for example, cylindrical shapes, can also modify release rates of a sustained release TCA/PLGA microparticle by virtue of the increased ratio of surface area to mass inherent to such alternative geometrical shapes, relative to a spherical shape. The microparticles have a volumetric mean diameter ranging between about 0.5 to 500 microns. In a preferred embodiment, the microparticles have a volumetric mean diameter of between 10 to about 100 microns.

Biodegradable polymer microparticles that deliver sustained release TCA may be suspended in suitable aqueous or non-aqueous carriers which may include, but is not limited to water, saline, pharmaceutically acceptable oils, low melting waxes, fats, lipids, liposomes and any other pharmaceutically acceptable substance that is lipophilic, substantially insoluble in water, and is biodegradable and/or eliminatable by natural processes of a patient's body. Oils of plants such as vegetables and seeds are included. Examples include oils made from corn, sesame, cannoli, soybean, castor, peanut, olive, arachis, maize, almond, flax, safflower, sunflower, rape, coconut, palm, babassu, and cottonseed oil; waxes such as carnoba wax, beeswax, and tallow; fats such as triglycerides, lipids such as fatty acids and esters, and liposomes such as red cell ghosts and phospholipid layers.

TCA Loading of and Release from Biodegradable PLGA Micronarticles

When intra-articularly delivered TCA is incorporated into a PLGA biodegradable polymer for sustained release into a joint at a dosage that does not suppress the HPA axis, preferred loadings of the TCA are between 22% to 28%, e.g., about 25%, (w/w) of the microparticle.

As the biodegradable PLGA polymers undergo gradual bio-erosion within the joint, the TCA is released to the inflammatory site. The pharmacokinetic release profile of TCA by the biodegradable PLGA polymer may be first order, zero order, bi- or multi-phasic, to provide desired treatment of inflammatory related pain. In any pharmacokinetic event, the bio-erosion of the polymer and subsequent release of TCA may result in a controlled release of TCA from the polymer matrix. The rate of release at dosages that do not suppress the HPA axis are described above.

### Excipients

The release rate of TCA from a PLGA biodegradable polymer matrix can be modulated or stabilized by adding a pharmaceutically acceptable excipient to the formulation. An excipient may include any useful ingredient added to the biodegradable polymer depot that is not a corticosteroid or a biodegradable polymer. Pharmaceutically acceptable excipients may include without limitation lactose, dextrose, sucrose, sorbitol, mannitol, starches, gum acacia, calcium phosphate, alginates, tragacanth, gelatin, calcium silicate, microcrystalline cellulose, PEG, polysorbate 20, polysorbate 80, polyvinylpyrrolidone, cellulose, water, saline, syrup, methyl cellulose, and carboxymethyl cellulose. An excipient for modulating the release rate of TCA from the biodegradable PLGA drug depot may also include without limitation pore formers, pH modifiers, solubility enhancers, reducing agents, antioxidants, and free radical scavengers.

### Delivery of TCA/PLGA Micronarticles

Parenteral administration of TCA/PLGA formulations of the invention can be effected by intra-articular injection or other injection using a needle. To inject the TCA/PLGA microparticles into a joint, needles having a gauge of about 14-28 gauge are suitable. It will be appreciated by those skilled in the art that TCA/PLGA formulations of the present invention may be delivered to a treatment site by other conventional methods, including catheters, infusion pumps, pens devices, injection guns and the like.

All references, patents, patent applications or other documents cited are hereby incorporated by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: Study Design

Intra-articular (IA) administration of triamcinolone acetonide injectable crystalline suspension (TAcs) is commonly used to treat pain and inflammation associated with osteoarthritis (OA) of the knee. The TCA/PLGA microparticles used in this study are an extended-release IA formulation of TCA at a load dose of between 22% to 28%, e.g., about 25%, (w/w) in 75:25 poly(lactic-co-glycolic acid) (PLGA) microspheres that is intended to deliver TCA to the synovial and peri-synovial tissues for a period of up to 3 months. The purpose of this study is a double-blind, randomized, single-dose, parallel group study to investigate the effects of an intra-articular injection of the TCA/PLGA microparticles on blood glucose in patients with osteoarthritis of the knee and type 2 diabetes.

The primary objective of this study is to assess the effects of a single intra-articular (IA) injection of 40 mg of TCA/PLGA microparticles, an extended release formulation of triamcinolone acetonide (TCA), on blood glucose concentrations in patients with type 2 diabetes, relative to 40 mg of TAcs.

The secondary objective of this study is to assess the safety and general tolerability of a single IA injection of 40 mg of TCA/PLGA microparticles relative to 40 mg of TAcs.

This study was a double-blind, randomized, parallel group, single dose design. The study was conducted in male and female patients ≧40 years of age with osteoarthritis (OA) of the knee and type 2 diabetes that did not require injectable agents to manage glucose. Patients must have been treated with 1 or 2 oral agents and had hemoglobin A1c (HbA1c) levels between 6.5%-9.0%. Patients who had been diagnosed with unilateral or bilateral OA of the knee, based on clinical and radiological criteria, and who were diagnosed with type 2 diabetes that did not require injectable agents to manage glucose were included in this study. In general, this population tolerates IA injections of commercially available corticosteroids (Habib, 2009). In prior clinical studies, 40 mg of TCA/PLGA microparticles administered as a single IA was well tolerated in this population.

Approximately 36 patients in total were randomized to one of two treatment groups (1:1) and treated with a single IA injection of either:
40 mg TCA/PLGA microparticles (18 pts)
40 mg TAcs (18 pts)

Blood glucose concentrations in each patient were evaluated for a total of 3 weeks (one week prior to injection and two weeks post injection). After a Screening visit, patients' blood glucose concentrations were measured using a Dexcom Z4 Platinum Professional™ continuous glucose monitoring (CGM) device, set to blinded mode, for up to 1 week pre-injection and for 2 weeks post injection using the same blinded CGM device. A final safety visit was planned at 6 weeks.

Approximately thirty-six patients were randomized and treated to ensure there were at least 30 evaluable patients (15 patients on TCA/PLGA microparticles and 15 patients on TAcs). As used herein, “TAcs” is a commercially available triamcinolone acetonide injectable crystalline suspension, 40 mg/mL, IA, administered as a 1 mL injection. As used herein, “TCA/PLGA microparticles” refer to an extended release formulation of TCA in 75:25 poly(lactic-co-glycolic) acid (PLGA) microspheres. Nominal 40 mg TCA, IA injection, were administered as a 5 mL injection.

To be included in the trial, patients must have fulfilled the following criteria: Written consent to participate in the study; Willingness and ability to comply with the study procedures and visit schedules and ability to follow verbal and written instructions; male or female >40 years of age; diagnosis of type 2 diabetes that does not require injectable agents (e.g. insulin or insulin analogs, exenatide, pramlintide, liraglutide) to manage glucose at least 1 year prior to screening; currently treated with at least one, but less than 3, oral agents for diabetes, with stable doses for at least 2 months; HbA1c≧6.5% and <9.0% at screening; symptoms associated with OA of the knee for at least 6 months prior to Screening (patient's report was acceptable); currently meets ACR Criteria (clinical and radiological) for OA for at least 6 months prior to Screening (American College of Rheumatology (ACR) Criteria for Classification of Idiopathic OA of the Knee including knee pain, osteophytes, and at least one of the following: age was greater than 50 years, stiffness for less than 30 minutes, and/or crepitus; index knee pain on most days (>15) over the last month (as reported by the patient); Body mass index (BMI)≦40 kg/m2; willingness to abstain from use of protocol-specified restricted medications during the study including the following: use of acetaminophen, or acetaminophen containing products; IA corticosteroids in any joint, IA viscosupplementation (hyaluronic acid) or any IA intervention (IA injection, IA aspiration, etc.) in the index knee, any investigational drug, device or biologic, immunomodulators, immunosuppressives, or chemotherapeutic agents, live or live attenuated vaccine, and/or IV, IM, oral, inhaled, intranasal or topical corticosteroids; willingness to wear, and remain within receiving range (no more than 20 ft.), a Continuing Glucose Monitoring (CGM) device uninterrupted for 24 hours per day throughout the study (3 weeks) and to comply with calibration requirements; must be accustomed to using a Standard Blood Glucose Measuring device by finger stick; adequate glucose data collected during the pre-treatment phase (Day −7 through Day −1). This includes at least one full day of data with no significant gaps.

Patients fulfilling at least one of the following disease-related criteria might not have been included in the study: Fibromyalgia, Reiter's syndrome, rheumatoid arthritis, psoriatic arthritis, ankylosing spondylitis or arthritis associated with inflammatory bowel disease; history of infection in the index knee joint; clinical signs and symptoms of active knee infection or crystal disease of the index knee within 1 month of screening; presence of surgical hardware or other foreign body in the index knee; unstable joint (such as a torn anterior cruciate ligament) within 12 months of screening.

Patients fulfilling at least one of the following previous or concomitant treatment-related criteria might not have been included in the study: IA corticosteroid (investigational or marketed) in any joint within 3 months of Screening; IA hyaluronic acid (investigational or marketed) in the index knee within 6 months of Screening: IV or IM corticosteroids (investigational or marketed) within 3 months of screening; oral, inhaled and intranasal corticosteroids (investigational or marketed) within 1 month of screening; inhaled, intranasal or topical corticosteroids (investigational or marketed) within 2 weeks of screening any other IA investigational drug/biologic within 6 months of screening; Prior use of TCA/PLGA microparticles; prior arthroscopic or open surgery of the index knee within 12 months of screening; planned/anticipated surgery of the index knee or any other surgery that would require use of a restricted medication during the study period; use of acetaminophen, or acetaminophen containing products, from screening through Day 15 (completion of post-treatment glucose monitoring).

Patients fulfilling at least one of the following patient-related criteria might not have been included in the study: Known hypersensitivity to any form of triamcinolone; History of sarcoidosis or amyloidosis; active of history of malignancy within the last 5 years, with the exception of resected basal cell carcinoma, squamous cell carcinoma of the skin, or effectively managed cervical carcinoma; known active or quiescent systemic fungal, bacterial (including tuberculosis), viral or parasitic infections, or ocular herpes simplex; any infection requiring intravenous antibiotics within 4 weeks of screening or infection requiring oral antibiotics within 2 weeks of screening; history of osteomyelitis; known or clinically suspected infection with human immunodeficiency virus (HIV), hepatitis B or C viruses; any clinically significant electrocardiogram (ECG) abnormality as judged by the Principal Investigator; patients requiring or likely to require treatment with corticosteroids during the study period based on patient medical history; current use of a continuous glucose monitoring device; history of or active Cushing's syndrome; active psychiatric disorder including psychosis (e.g. schizophrenia), bipolar disorder, uncontrolled anxiety disorder and major depressive disorder; active substance abuse (drugs or alcohol), history of chronic substance abuse within the last year, or prior chronic substance abuse judged by the investigator likely to recur during the study; skin breakdown at the knee where the injection would take place; women who were pregnant, nursing or plan to become pregnant during the study or through Week 6 if discontinued early; men whose partners plan to attempt conception during the study or through Week 6 if discontinued early; women of child-bearing potential (not surgically sterile or post-menopausal for at least 1 year as documented in medical history) not using a highly effective method of contraception (oral, injected or implanted hormonal methods of contraception; intrauterine device (IUD) or intrauterine system (IUS); condom or occlusive cap (diaphragm or cervical/vault caps) with spermicidal foam/gel/film/cream/suppository; or male sterilization (vasectomy)); use of immunomodulators, immunosuppressives, or chemotherapeutic agents within 5 years of screening; has received a live or live attenuated vaccine within 3 months of screening; use of any other investigational drug or device within 30 days of screening or within 5 half-lives (whichever was longer) or an investigational biologic within 60 days of screening or within 5 half-lives (whichever was longer); any other clinically significant acute or chronic medical conditions that, in the judgment of the Investigator, would preclude the use of an IA corticosteroid or that could compromise patient safety, limit the patient's ability to complete the study, and/or compromise the objectives of the study.

The study involved the following: A Screening visit, a Pre-treatment visit, (˜1 week prior to Day 1) where the CGM sensor were placed, and a Pre-treatment phase (a one week period where CGM data were collected). Randomization and the IA injection took place on Day 1 and the Follow-up phase consisted of 2 additional visits at Days 8 and 15. CGM data continued to be collected until the sensor was removed at Day 15. A final FU visit occurred approximately 6 weeks after study entry at Day 43.

At the screening visit patients provided informed consent, underwent a physical examination, blood samples were collected for laboratory safety tests, an ECG and vital signs were collected or measured. Information regarding medical history and the use of pre-trial medication were collected. OA medical history included ACR diagnosis details, OA diagnosis date (if available), Kellgren-Lawrence (K-L) grade (if available), number of days with index knee pain in the last month, previous IA steroid or hyaluronic injections, presence of OA in other joints, prior procedures or surgeries for OA.

After the screening visit, the subject returned to the clinic for the pre-treatment study visit where the CGM sensor were placed. At this visit, patients were fitted with and instructed on the use of the Dexcom G4 Platinum Professional™ CGM device in blinded mode and were provided a Bayer Contour™ Standard Glucose Measuring meter and testing strips in order to perform the required calibrations. Calibration requirements of the CGM were based on the manufacturer's recommendation. During the approximately one week pre-treatment phase of the study, blood glucose was continuously measured using the CGM device.

Following confirmation that all entry criteria have been met and after review of the glucose data by a third party vendor confirmed there was adequate data to continue (there must have been at least one full day of data with no significant data gaps, as well as confirmation that all necessary calibrations were performed) patients were then randomized to one of 2 treatment groups on Day 1. Patients were fitted with a replacement sensor and received a single IA injection in their index knee at approximately 3 hours after sensor placement.

If a subject returned on Day 1 and was found to have had insufficient glucose data (i.e. not all calibrations performed or data collection does not meet the above criteria), a subject may have repeated the full 7 day pre-treatment phase at the discretion of the Investigator in collaboration with the Medical Monitor. Patients were given just one opportunity to repeat the pre-treatment phase.

Following treatment, patients returned at Day 8 and underwent CGM sensor replacement, a physical examination and an index knee assessment were performed and vital signs were collected. The index knee assessment was performed by the designated assessor at the days indicated in the Schedule of Assessments. The index knee was assessed for tenderness, heat/redness, swelling, effusion, and Baker's cyst. If there were a clinically significant finding at the Screening or Baseline Visit, it was added to the Medical History. At time points post-baseline, if there were new clinically significant findings or findings that worsen for the patient's baseline condition, they were recorded as AEs. Information regarding adverse events (AEs) and concomitant medications was also collected. On Day 15 Patients had the CGM device removed and vital signs were collected, and an index knee assessment was performed. Information regarding AEs and concomitant medications was also collected.

Patients returned for a follow-up visit 6 weeks following treatment, at Day 43. Patients underwent a physical examination, vital signs were collected, and an index knee assessment was performed. Information regarding AEs and concomitant medications was also collected. Patients were required to monitor their blood glucose concentrations continually through Day 15.

Intra-articular Injection Procedure:

IA injections were performed by the assigned unblinded injector, who had significant experience in the administration of IA injections. The injector had discretion to choose the position of the knee (e.g., extended or bent), the approach for the injection (e.g., medial or lateral) and the numbing agent used (ethyl chloride or subcutaneous lidocaine only; IA anesthetics were not allowed) based on standard of care. Sterile technique was used.

Prior to injection, the index knee was thoroughly cleansed using a bactericidal solution. The index knee was aspirated in all cases prior to administration of study medication. Following aspiration, 5 mL of the reconstituted TCA/PLGA microparticles or 1 mL of TAcs were injected into the synovial space. The injection contents were not visible to the patient (e.g., covering syringe or using a screen).

The same needle used for IA injection of the study medication could also have been used for synovial fluid aspiration, thereby allowing for a single injection with syringe replacement. The injector used a 21 gauge needle or larger for injection and aspiration of fluid.

The blinded assessor was not present during the administration of study medication but was available immediately thereafter to assess any immediate safety events that may emerge.

Timing of the injection took place approximately 3 hours following the sensor placement on Day 1. The IA injection took place once the sensor had been calibrated (approximately 3 hours after placement).

Patients were advised to avoid strenuous activities or prolonged weight-bearing activities for approximately 24 to 48 hours following the injection.

In the event that the patient had an immediate reaction (e.g., tenderness, increased pain, swelling, effusion, decreased mobility of the index knee), the patient was treated according to local clinical guidelines and physician experience.

Concomitant Medications:

Restricted medications/therapies during the study included: acetaminophen or acetaminophen containing medications, oral, inhaled or intranasal corticosteroids, IA corticosteroids in any joint, IA viscosupplementation (hyaluronic acid) or any IA intervention (IA injection, IA aspiration, etc.) in the index knee, any investigational drug, device or biologic, immunomodulators, immunosuppressives, or chemotherapeutic agents, live or live attenuated vaccines and other investigational therapies (drug, biologic or device). Washout periods for these medications/therapies were defined in the Exclusion criteria.

Patients may have taken analgesic medications including non-steroidal anti-inflammatory drugs, tramadol and opioids, except those containing acetaminophen (examples: Tylenol™, Tylenol™ cold remedies, Nyquilm), as needed or prescribed by the investigator.

Patients were advised to maintain a stable lifestyle with regard to physical activity throughout the duration of the study.

Pharmacodynamic properties were evaluated by measuring blood glucose concentrations. Safety was evaluated by measuring adverse events (AEs); physical examinations; index knee examinations; vital signs; and/or clinical laboratory evaluations.

Statistical Methods:

Descriptive statistics (n, mean, Standard Deviation (SD), median, minimum, maximum) were calculated by treatment group and time point for continuous variables. Frequencies and percentages were presented by treatment group for categorical and ordinal variables. All study data were standardized into CDISC compliant SD™ and ADaM datasets, and all data collected were presented in patient listings.

Two study populations were planned for this study: A safety population which included all subjects who provide informed consent and were treated with randomized study medication. Patients in the safety population were analyzed as treated. A full-analysis set (FAS) population included all patients who receive randomized treatment and provided sufficient CGM monitoring information to contribute to planned analyses. Inclusion into the FAS was completed by blinded review of CGM data and patients included in the FAS were analyzed as randomized.

Safety analyses were performed on the Safety Population. Safety and tolerability were evaluated on the basis of AEs spontaneously reported by the patient or discovered by the Investigator and findings from the following assessments: physical examinations, index knee assessments, vital signs, and clinical laboratory evaluations.

AEs were coded using the Medical Dictionary for Regulatory Activities (MedDRA) dictionary. Incidences (number and percent) of treatment-emergent adverse events (TEAEs), those events that started after dosing or worsened in severity after dosing, were presented by treatment group. Incidences of TEAEs were also presented by maximum severity and relationship to study medication. Safety laboratory investigations ECGs, and vital signs were summarized descriptively by time point collected, and with changes from baseline (screening) assessment between dosing and the week 6 visit.

The primary endpoint was the average glucose over time from Baseline over 72 hours post injection. Average daily glucose was calculated from time points midnight (00:00) to 23:59. Average glucose over time from baseline to 72 hours was analyzed with a mixed model for repeated measures (MMRM) with fixed effects for treatment group and the mean of the pre-treatment glucose values as the baseline covariate, and treatment×time interaction terms. Other baseline covariates (e.g. Age, Gender, BMI, Body Weight at baseline) were explored for inclusion in the mixed models. Least square means (LSMeans), 95% confidence intervals, and p-values for individual treatments and treatment differences were presented for each model.

The secondary endpoints included the following: (i) Mean blood glucose concentrations measured using a CGM over 7 and 14 days post IA injection for TCA/PLGA microparticles relative to TAcs were analyzed with a mixed model for repeated measures (MMRM), similar to that for the primary endpoint; (ii) Percent of time blood glucose less than 70, between 70 and 180, greater than 180 and greater than 250 mg/dL in the first 24 hours post injection, 1 week and 2 weeks post injection; (iii) Glycemic variability (CV) in the first 24 hours post injection, over 1 week and 2 weeks post injection; and (iv) Glycemic variability (SD) in the first 24 hours post injection over 1 week and 2 weeks post injection: (a) Distance Traveled in the 72 hours post injection, over 1 week and 2 weeks post injection, and (b) Energy in the 72 hours post injection, over 1 week and 2 weeks post injection.

Other suitable analyses and evaluations included, by way of non-limiting examples, median time to earliest significantly increased blood glucose concentrations for TCA/PLGA microparticles and TAcs, where a significant increase in blood glucose concentration after the injection was defined as the first concentration observed that was higher by at least 2 standard deviations (SDs) than the mean blood glucose concentration at baseline before the injection (see e.g., Habib 2009)); time to peak blood glucose concentrations following IA injection; cumulative distribution of the post-treatment daily average blood glucose over 2 weeks post IA injection; and/or maximum blood glucose concentrations for TCA/PLGA microparticles and TAcs.

### Example 2: Interim Evaluation of Effects of TCA/PLGA Microparticles on Blood Glucose Levels in Patients with Type 2 Diabetes Mellitus

As noted previously, approximately 14.4% of patients with OA have been diagnosed with DM (Louati et al, 2015). In this population, IA corticosteroids can increase blood glucose (BG) for approximately 72 hours post-injection (Habib et al, 2008). Because systemic exposure to TCA produced by TCA/PLGA microparticles is reduced relative to TAcs, it is plausible that the evaluation in BG observed following injection of TAcs will be reduced following injection of TCA/PLGA microparticles.

The primary objective of this study was the assessment of the effects of a single IA injection of 40 mg of the TCA/PLGA microparticles on BG levels in patients with type 2 diabetes mellitus (DM) relative to 40 mg of TCA in a other standard triamcinolone acetonide (TA) crystalline suspension (“TAcs”). The design of this study is presented in Example 1.

One group of patients received a single 5 mL intraarticular (IA) injection of 40 mg of an extended release TCA/PLGA microparticle formulation in which TCA is formulated in 75:25 PLGA microspheres. A second group of patients received a single 1 mL IA injection of a commercially available, non-extended release formulation of TCA, referred to herein as TCA Immediate Release or TAcs. Patients were randomized into these groups in a 1:1 ratio. The randomization was performed across study centers employing a minimization procedure to minimize baseline average blood glucose concentration difference by CGM.

A total of 33 patients were randomized in the study, with 17 in the 40 mg TCA/PLGA microparticles group and 16 in the TAcs group. Three patients were randomized correctly, but received the incorrect treatment assignment, and, therefore, 18 patients received the TCA/PLGA microparticles and 15 patients received TAcs.

A total of 33 patients completed the study through Day 15, and 32 patients completed the study through Week 6 (safety follow-up).

Review of demographic and baseline characteristics by treatment group showed that overall, the treatment groups were reasonably balanced. Overall, among all 33 patients, 14 were female and 19 were male. The mean age at consent was 61 years, with a range of 46 to 83 years. Most patients were white (24/33); of the remaining patients, 8 were black/African-American; and 1 was Asian. Mean height, weight, and BMI were 171.8 cm, 98.1 kg, and 33.3 kg/m2, respectively. Based on BMI, most (24/33) patients were obese. Baseline average blood glucose categories were not balanced amongst treatment groups in the FAS due to the error with the incorrect treatment assignment for 3 patients. There were more patients in the 157.33-177.43 mg/dL category treated with TCA/PLGA microparticles (n=5) than with TAcs (n=1).

All 33 (100%) patients had a diagnosis of OA of the knee, as confirmed per ACR criteria, in accordance with the protocol entrance criteria. The mean time since primary diagnosis was 9.2 years, with a wide range of 1.0 to 51.0 years. Overall, 57.6% (19/33) of patients had bilateral knee OA, with 42.4% (14/33) of patients having unilateral knee OA. The mean number of days with knee pain within the month prior to Screening was 25. Most (72.7%; 24/33) patients had a K-L grade of 2, with 2 (6.1%) patients each having a K-L grade of 3 or 4 and 1 (3.0/%) having a K-L grade of 1. The K-L grade was unknown for the remaining 4 (12.1%) patients.

Overall, approximately one-third (30.3%; 10/33) of patients had received a prior IA steroid injection in the index knee, with the maximum number of such treatments being 4, and 9.1% (3/33) patients had received a prior HA injection in the index knee, with the maximum number of such treatments being 3. A total of 6 (18.2%) patients had undergone prior surgery or procedures to treat OA in the index knee.

Nineteen (57.6%) patients had a history of OA in a location other than the knee, most commonly in the lumbar spine or hip (each 7 patients; 21.2%), shoulder (6 patients; 18.2%), or hand (5 patients; 15.2%).

The diabetes history for these patients was recorded. The mean duration since type 2 DM diagnosis was 8.6 years, with a range of 1 to 22 years, overall, with mean durations of 7.8 years (range 2 to 22 years) and 9.6 years (range 1 to 21 years) in the TCA/PLGA microparticles and TAcs groups, respectively. No patient had a history of diabetic ketoacidosis. Five (15.2%) patients had a history of prior insulin use.

Review of OA history and index knee characteristics by treatment group revealed some variation, which is not unexpected given the relatively small sample size in each treatment group. As efficacy was not an objective of this study, these variations were not of concern.

The primary endpoint was the change in average blood glucose from baseline (Hour −72 to Hour −1) to Day 1-3 (Hour 1 to Hour 72) for the TCA/PLGA microparticles 40 mg relative to TAcs 40 mg. The primary analysis was completed using a linear model (ANCOVA) with fixed effects for treatment group. Model covariates were study site and baseline (72-hour) blood glucose average. The area under the effect (AUE) curve for average blood glucose over various time intervals was examined as a secondary endpoint. AUE was calculated from a linear trapezoidal rule. Exploratory pharmacodynamic endpoints were also analyzed. These data are shown in Tables 1-5 and described in more detail below.

TABLE 1
Analysis of Change from Baseline for Average Blood Glucose (mg/dL) (−72
Hours to 72 Hours), by Treatment Group (FAS; N = 33)

| | TCA/PLGA Microspheres 40 mg | | TAcs 40 mg | |
|---|---|---|---|---|
| | (N = 18) | | (N = 15) | |
| | | Change From | | Change From |
| Timepoint/Statistic | Observed | Baseline | Observed | Baseline |
| Baseline | | | | |
| (Hour −72 to Hour −1) | | | | |
| n | 18 | | 15 | |
| Mean (SD) | 155.24 (38.203) | | 161.72 (40.512) | |
| Median | 149.39 | | 150.94 | |
| Min; Max | 96.5; 233.9 | | 103.9; 266.8 | |
| Day 1-3 | | | | |
| (Hour 1 to Hour 72) | | | | |
| n | 18 | 18 | 15 | 15 |
| Mean (SD) | 163.41 (52.141) | 8.17 (25.999) | 198.78 (56.241) | 37.07 (25.152) |
| Median | 146.76 | −1.45 | 182.10 | 31.16 |
| Min; Max | 89.8; 298.8 | −21.0; 72.5 | 135.1; 315.8 | −4.9; 89.6 |
| LSM (SE) | | 14.66 (7.032) | | 33.88 (6.612) |
| 95% CI | | 0.14, 29.17 | | 20.24, 47.54 |
| LSM Difference from TAcs | | −19.23 | | |
| 95% CI | | −38.01, −0.44 | | |
| 2-sided p-value | | 0.0452 | | |

The primary endpoint for the change in average daily blood glucose from baseline (Hour −72 to Hour −1) to Day 1-3 (Hour 1 to Hour 72) was met for TCA/PLGA microparticles versus TAcs. TCA/PLGA microparticles 40 mg demonstrated a statistically significantly smaller LSM change in average daily blood glucose from baseline to Day 1-3 at 14.66 (7.032) compared with TAcs 40 mg at 33.89 (6.612). The LSM difference from TAcs was statistically significant (2-sided p-value: 0.0452) with a difference of −19.23 mg/dL and 95% CI (−38.01, −0.44).

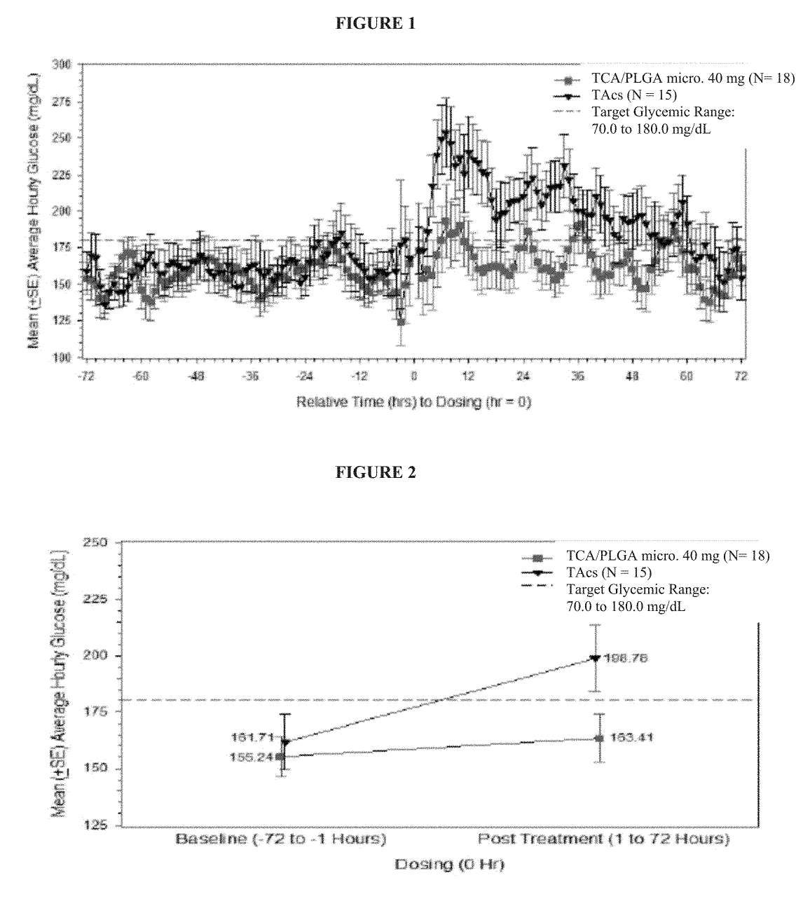

The mean average hourly blood glucose and the mean change from baseline to Day 1-3 for the average blood glucose is displayed in FIG. 1 and FIG. 2, respectively.

Mean average daily blood glucose had a small increase of 8.17 mg/dL for TCA/PLGA microparticles-treated patients from baseline to Day 1-3 (155.24, baseline to 163.41, Day 1-3). Conversely, mean average daily blood glucose increased by 37.07 mg/dL (161.71, baseline to 198.78, Day 1-3) over the same time period for TAcs treated patients. The difference between the two groups is 28.9 mg/dL, which is associated with a clinically meaningful difference between TCA/PLGA microparticles and TAcs (FIG. 2).

A post-hoc analysis was conducted to explore earlier effects on average daily blood glucose from baseline (Hour −48 to Hour −1) to Day 1-2 (Hour 1 to Hour 48). The LSM change in average daily blood glucose from baseline (Hour −48 to Hour −1) to Day 1-2 (Hour 1 to Hour 48) is displayed in Table 2.

TABLE 2
Analysis of Change from Baseline for Average Blood Glucose
(mg/dL) (−48 Hours to 48 Hours) (FAS; N = 33)

| | TCA/PLGA Microspheres 40 mg | | TAcs 40 mg | |
|---|---|---|---|---|
| | (N = 18) | | (N = 15) | |
| | | Change From | | Change From |
| Time point/Statistic | Observed | Baseline | Observed | Baseline |

Baseline (Hour −48
to Hour −1)

| n | 18 | | | | 15 | | | |
|---|---|---|---|---|---|---|---|---|
| Mean (SD) | 156.55 | (40.644) | | | 163.67 | (45.952) | | |
| Median | 150.61 | | | | 145.98 | | | |
| Min, Max | 86.8; 237.8 | | | | 103.3; 298.1 | | | |
| Day 1-2 (Hours 1 | | | | | | | | |
| to Hour 48) | | | | | | | | |
| N | 18 | | 18 | | 15 | | 15 | |
| Mean (SD) | 166.09 | (53.066) | 9.53 | (28.156) | 210.44 | (58.272) | 46.77 | (33.447) |

| Median | 146.31 | 0.16 | | 192.60 | 41.28 | |
|---|---|---|---|---|---|---|
| Min; Max | 95.9; 308.2 | −23.8; 70.4 | | 140.3; 325.2 | −4.2; 112.3 | |
| LSM (SE) | | 16.33 | (8.791) | | 43.62 | (8.255) |

| 95% CI | −1.82, 34.47 | 26.58, 60.66 |
|---|---|---|
| LSM Difference from TAcs | −27.30 | |
| 95% CI | −50.79, −3.80 | |
| 2-sided p-value | 0.246 | |

TCA/PLGA microparticles 40 mg demonstrated a significantly smaller LSM change in average daily blood glucose from baseline to Day 1-2 at 16.33 (8.791) compared with TAcs 40 mg at 43.63 (8.255). The LSM difference from TAcs was statistically significant (2-sided p-value: 0.0246) with −27.3 mg/dL and 95% CI (−50.80, −3.80).

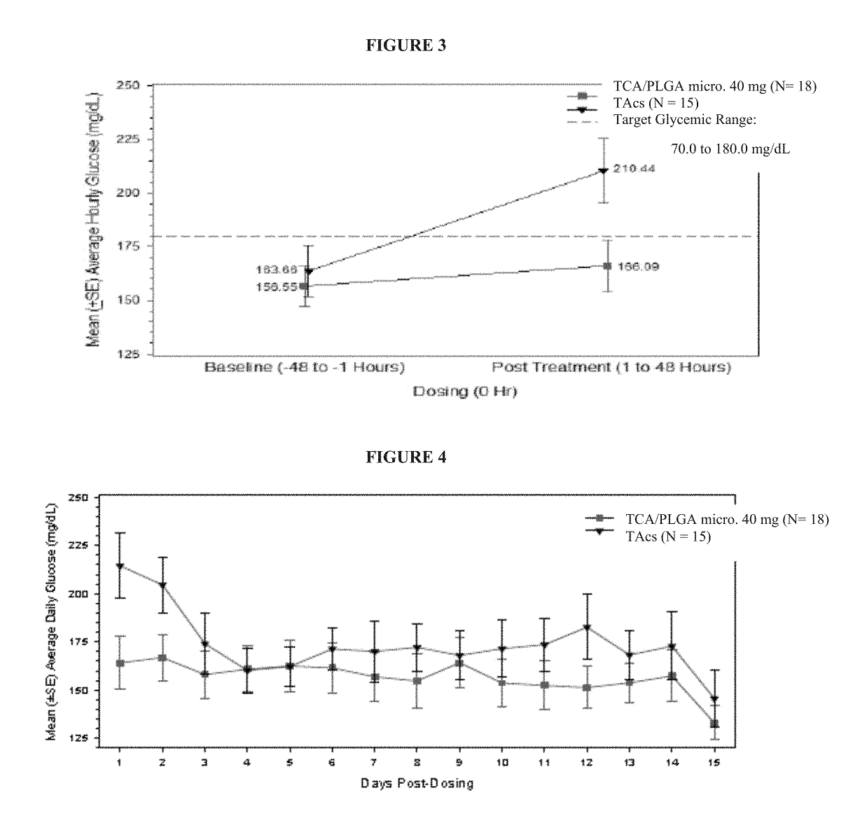

The mean change from baseline to Day 1-2 for the average blood glucose is displayed in FIG. 3.

Mean average blood glucose had a small increase of 9.54 mg/dL for TCA/PLGA microparticles-treated patients from baseline to Day 1-2 (156.55, baseline to 166.09, Day 1-2). Conversely, mean average blood glucose increased by 46.78 mg/dL (163.66, baseline to 210.44, Day 1-2) over the same time period for TAcs treated patients. The difference between the two groups is 37.24 mg/dL which is associated with a clinically meaningful difference between TCA/PLGA microparticles and TAcs (FIG. 3). These data suggest that the perturbation of glucose control produced by TAcs is maximal during the first 48 hours post-injection.

The mean average daily glucose by day over the 15-day post-injection glucose monitoring period is displayed in FIG. 4.

Over the entire time course of the 15-day post injection glucose monitoring period, blood glucose levels associated with TCA/PLGA microparticles remained at or below those produced by TAcs.

TABLE 3
Analysis of Area under the Effect Curve (AUE) for Average
Blood Glucose (mg/dL) Over Days 1-3 (FAS; N = 33)

| | | Treatment Group | |
|---|---|---|---|
| | | TCA/PLGA | |
| | | Microspheres 40 mg | TAcs 40 mg |
| AUE | Statistic | (N = 18) | (N = 15) |
| Day 1 | LSM (SE) | 3703.3 (239.62) | 4483.2 (225.30) |
| (Hours | 95% CI | 3208.8, 4197.9 | 4018.2, 4948.2 |
| 0-24) | LSM Difference | −779.8 | |
| | from TAcs | | |
| | 95% CI | −1420.0, −139.7 | |
| | 2-sided p-value | 0.0190 | |
| Days 1-2 | LSM (SE) | 7902.6 (442.21) | 9251.7 (415.78) |
| (Hours | 95% CI | 6989.9, 8815.3 | 8393.6, 10109.9 |
| 0-48) | LSM Difference | −1349.2 | |
| | from TAcs | | |
| | 95% CI | −2530.5, −167.8 | |
| | 2-sided p-value | 0.0269 | |
| Days 1-3 | LSM (SE) | 11772.3 (652.17) | 12950.9 (613.20) |
| (Hours | 95% CI | 10426.3, 13118.4 | 11685.3, 14216.4 |
| 0-72) | LSM Difference | −1178.5 | |
| | from TAcs | | |
| | 95% CI | −2920.8, 563.8 | |
| | 2-sided p-value | 0.1755 | |

Over the period of 0-72 hours (Days 1-3), the AUE for average BG was lower with 40 mg TCA/PLGA microparticles versus 40 mg TAcs, with an LSM difference between groups of −1178.5; however, the difference between groups was not statistically significant.

The AUE for average BG was statistically significantly lower with 40 mg TCA/PLGA microparticles versus 40 mg TCA over the time interval of 0-24 hours (Day 1) (LSM [SE] 3703.3 [239.62] and 4483.2 [225.30], respectively), with an LSM difference between 40 mg TCA/PLGA microparticles and 40 mg TAcs of −779.8 (95% CI −1420.0; −139.7; p=0.0190). The AUE for average BG was also statistically significantly lower with 40 mg TCA/PLGA microparticles versus 40 mg TAcs over the time interval of 0-48 hours (Days 1-2) (LSM [SE] 7902.6 [442.21] and 9251.7 [415.78], respectively), with an LSM difference between 40 mg TCA/PLGA microparticles and 40 mg TAcs of −1349.2 (95% CI −2530.5, −167.8; p=0.0269).

The time in the glycemic target range (70.0 to 180.0 mg/dL) was greater for TCA/PLGA microparticles compared to TAcs (64.1% vs. 42.7%, respectively) over 48 hours post-injection. The time above the glycemic target range (>180 mg/dL) was less for TCA/PLGA microparticles compared to TAcs (34.6% vs. 57.0%, respectively) over the same time period.

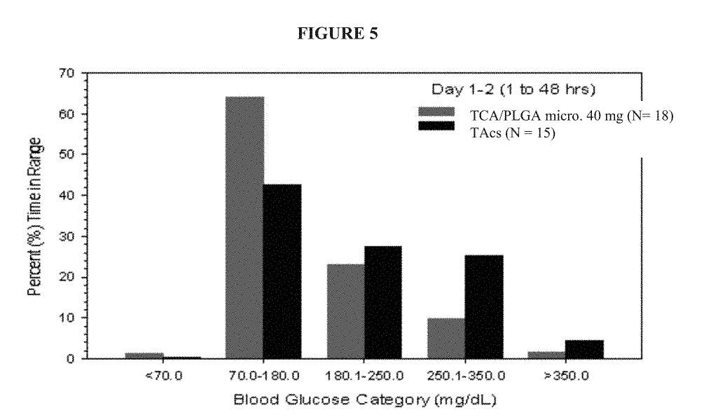

The average daily blood glucose by category for Day 1-2 is shown in FIG. 5.

TABLE 4
Analysis of Glycemic Variability (FAS; N = 33)

Treatment Group

| | TCA/PLGA | |
|---|---|---|
| | Microspheres 40 mg | TAcs 40 mg |
| Timepoint/Statistic¹ | (N = 18) | (N = 15) |
| Hours 1-72 | | |
| N | 18 | 15 |
| Mean (SD) | 24.03 (4.575) | 26.69 (6.418) |
| Median | 24.63 | 27.15 |
| Min; Max | 15.7; 31.7 | 16.8; 38.7 |
| LSM (SE)² | 22.43 (1.434) | 27.05 (1.349) |
| 95% CI | 19.47, 25.39 | 24.27, 29.83 |
| LSM Difference | −4.62 | |
| from TAcs | | |
| 95% CI | −8.45, −0.78 | |
| 2-sided p-value | 0.0202 | |

A statistically significant difference was seen between 40 mg TCA/PLGA microparticles and 40 mg TAcs with regard to the degree of glycemic variability over the time period of 1-72 hours, with less variability seen with 40 mg TCA/PLGA microparticles than with 40 mg TAcs (LSM [SE] 22.43 [1.434] versus 27.05 [1.349]; LSM difference from TAcs −4.62, 95% CI −8.45, −0.78; p=0.0202). For all other time periods assessed, no statistically significant differences between groups were seen with regard to the degree of glycemic variability.

An additional analysis of change from baseline in the degree of glycemic variability also revealed significantly less variability relative to baseline (Hour −72 to Hour −1) with 40 mg TCA/PLGA microparticles than with 40 mg TAcs over the time period of 1-72 hours (LSM difference from TCA IR −4.23; 95% CI −7.81, −0.65; p=0.0225).

Analysis of the distance traveled (i.e., maximum hourly BG value −72-hour baseline BG value) for average hourly BG showed that the distance traveled was shorter in the 40 mg TCA/PLGA microparticles group than in the 40 mg TAcs group in each of the time periods assessed, indicating less fluctuation in glucose levels in the 40 mg TCA/PLGA microparticles group than in the 40 mg TAcs group.

TABLE 5
Analysis of Change from Baseline for Maximum Blood Glucose (mg/dL) (FAS; N = 33)

| | TCA/PLGA Microspheres 40 mg | | TAcs 40 mg | |
|---|---|---|---|---|
| | (N = 18) | | (N = 15) | |
| | | Change From | | Change From |
| Timepoint/Stastistic | Observed | Baseline | Observed | Baseline |
| Baseline average blood | | | | |
| glucose (Hour −72 to Hour −1) | | | | |
| N | 18 | | 15 | |
| Mean (SD) | 155.24 (38.203) | | 161.72 (40.512) | |
| Median | 149.39 | | 150.94 | |
| Min; Max | 96.5; 233.9 | | 103.9; 266.8 | |
| Day 1-3 maximum blood | | | | |
| glucose (Hour 1 to Hour 72) | | | | |
| N | 18 | 18 | 15 | 15 |
| Mean (SD) | 255.36 (66.544) | 100.12 (35.577) | 320.38 (56.007) | 158.66 (39.813) |
| Median | 249.71 | 92.27 | 334.88 | 169.14 |
| Min; Max | 153.7; 389.8 | 53.7; 163.5 | 206.0; 401.0 | 62.2; 235.4 |
| LSM (SE) | | 110.04 (10.084) | | 158.46 (9.481) |
| 95% CI | | 89.23, 130.85 | | 138.89, 178.03 |
| LSM Difference from TAcs | | −48.42 | | |
| 95% CI | | −75.36, −21.48 | | |
| 2-sided p-value | | 0.0011 | | |

As shown, 40 mg TCA/PLGA microparticles demonstrated a statistically significantly smaller LSM change from baseline average BG to maximum BG over Day 1-3 at 110.04 (10.084) compared with 40 mg TAcs at 158.46 (9.481). The LSM difference from TAcs of −48.42 mg/dL (95% CI: −75.36, −21.48) was statistically significant (p=0.0011).

Mean (SE) maximum daily BG values over the 15-day post injection glucose monitoring period correlates with the daily averages.

During the time period of hours 1-72 (Days 1-3), the median time to the first significantly increased BG value was statistically significantly longer in the 40 mg TCA/PLGA microparticles group than in the 40 mg TAcs group (31 hours versus 5 hours, respectively; p=0.0068).

During the time period of hours 1-72 (Days 1-3), the median time to the maximum hourly average BG value was significantly longer in the 40 mg TCA/PLGA microparticles group than in the 40 mg TAcs group (34 hours versus 13 hours, respectively; p=00.0071.

A single IA injection of 40 mg TCA/PLGA microparticles or 40 mg TAcs was well tolerated in this study. Review of TEAEs showed that: (i) The overall incidence of TEAEs overall was low (11.10% [2/18] in the 40 mg TCA/PLGA microparticles group and 13.3% [2/15] in the 40 mg TAcs group); (ii) No particular TEAE was reported by >1 patient; (iii) All TEAEs were assessed by the Investigator as Grade 1 or 2 in severity; (iv) No TCA/PLGA microparticles-treated patient experienced an index knee TEAE or TEAE related to the injection procedure. One TAcs-treated patient experienced an index knee TEAE that was related to the injection procedure and study drug (Grade 1 ecchymosis at the injection site); no other index knee TEAEs or TEAEs related to the injection procedure were reported among TAcs-treated patients; (v) No deaths or other SAEs were reported; and (vi) No patient discontinued from the study because of a TEAE.

Disruption of glucose control following IA corticosteroid injections can present a problem for patients with type 2 DM. The results of this study demonstrate that over 48 and 72 hours post IA injection, treatment with TCA/PLGA microparticles did not significantly disrupt the glycemic control of subjects with type 2 DM managed with 1 to 2 oral drugs. In contrast, TAcs resulted in disruption of glycemic control, consistent with prior literature (FIG. 2, FIG. 3, Habib et al, (2008) Increased blood glucose levels following intra-articular injection of methylprednisolone acetate in controlled diabetic patients with symptomatic osteoarthritis of the knee. Ann Rheum Dis 2008; 67:1790-1791.).

The method used to monitor blood glucose in this study was robust. The Dexcom Z4 Platinum Professional™ CGM uses the same technology as the Dexcom G4 Platinum, shown to be highly accurate with an average error of ˜11% (Damiano et al, A Comparative Effectiveness Analysis of Three Continuous Glucose Monitors: The Navigator, G4 Platinum, and Enlite. J Diabetes Sci Technol. 2014; 99 (5): 1701-1711.). The CGM device was calibrated with the Bayer Contourmi Next standard glucose measuring meter, one of the most accurate glucose meters available with an average error of ˜6% (Ekhlaspour et al, Comparative Accuracy of 17 Point-of-Care Glucose Meters. J Diabetes Sci Technol. 2015). The CGM technology has the advantage of capturing a comprehensive representation of glucose variability over the entire day and night, which is not possible using spot point-of-care testing.

Disruption of glycemic control following IA corticosteroid injections represents a clinical problem for diabetic patients. Although the disruption is transient, it can be associated with concerning symptoms, including blurred vision, increased urination, increased thirst, and fatigue. In this study, patients who received TAcs had peak glucose levels on average over the glycosuric threshold of 200 mg/dL, and could have been expected to suffer from these signs and symptoms (Butterfield et al, Renal glucose threshold variations with age. Br Med J. 1967; 4(5578):505-507).

The time in glycemic target range (70-180 mg/dL) (American Diabetes Association, 2016) was greater for TCA/PLGA microparticles as compared to TAcs over the 48 hours post IA injection, providing another indication of the improvement in glycemic control. In clinical practice, changes made to patients' diabetes regimens in response to steroid-induced hyperglycemia that can subsequently lead to hypoglycemia once the effects of the corticosteroids have resolved. Such untoward and potentially harmful management is less likely in the absence of disruptions in glycemic control.

Glycemic variability associated with TCA/PLGA microparticles was also significantly reduced relative to TAcs in the first 72 hours post-injection. Although glycemic variability has not been definitively linked to diabetes complications, there is a biological rationale (Esposito et al, Postprandial Hyperglycemia Study Group. Regression of carotid atherosclerosis by control of postprandial hyperglycemia in type 2 diabetes mellitus. Circulation 2004; 110:214-219; Kilpatrick et al. The effect of glucose variability on the risk of microvascular complications in type 1 diabetes. Diabetes Care 2006; 29:1486-1490) supporting glycemic variability as a risk factor in the pathogenesis of the vascular complications, and elevated glycemic variability is associated with hypoglycemia (Qu et al, Rate of hypoglycemia in insulin-treated patients with type 2 diabetes can be predicted from glycemic variability data. Diabetes Technol Ther 2012; 14(11)1008-1012), reduced patient satisfaction (Testa et al, Comparative effectiveness of basal-bolus versus premix analog insulin on glycemic variability and patient-centered outcomes during insulin intensification in type 1 and type 2 diabetes: a randomized, controlled, crossover trial. J Clin Endocrinol Metab 2012; 97:3504-3514), and biochemical abnormalities possibly linked to diabetes complications (Wentholt et al, Glucose fluctuations and activation of oxidative stress in patients with type 1 diabetes. Diabetologia 2008; 51:183-190).

Over the entire time course of the 15-day post injection glucose monitoring period, blood glucose levels associated with TCA/PLGA microparticles remained at or below those produced by TAcs. This observation is consistent with PK studies demonstrating low systemic exposure to TCA associated with TCA/PLGA microparticles.

The evaluation of safety data from 33 patients treated with a single IA injection of either 40 mg TCA/PLGA microparticles (N=18) or 40 mg TAcs (N=15) suggest that both TCA/PLGA microparticles and TAcs were well tolerated. No new or unexpected safety concerns were identified with 40 mg TCA/PLGA microparticles in this study.
There were no deaths or other SAEs reported on study.
No patient discontinued from the study due to a TEAE.
The overall incidence of TEAEs overall was low (11.1% [2/18] in the 40 mg TCA/PLGA microparticles group and 13.3% [2/15] in the 40 mg TAcs group).
All TEAEs were Grade 1 or Grade 2 in severity.
No particular TEAE was reported by >1 patient.
No TCA/PLGA microparticles-treated patient experienced an index knee TEAE or TEAE related to the injection procedure. One TAcs-treated patient experienced an index knee TEAE that was related to the injection procedure and study drug, Grade 1 ecchymosis at the injection site; no other index knee TEAEs or TEAEs related to the injection procedure were reported among TAcs-treated patients.
Analyses of changes from Baseline for hematology and chemistry parameters did not reveal any remarkable trends.
No clinically relevant changes in vital signs attributable to TCA/PLGA microparticles or TAcs were observed.

Safety findings from this study support administration of a single IA injection of 40 mg TCA/PLGA microparticles in patients with OA of the knee. No new or unexpected safety concerns were identified.

Treatment with TCA/PLGA microparticles 40 mg resulted in a significantly (p=0.0452) smaller increase in blood glucose relative to TAcs over a 72-hour period following IA injection. As well, the time in glycemic target range (70-180 mg/dL) was greater for TCA/PLGA microparticles as compared to TAcs over the 48 hours post IA injection. These findings are consistent with the relatively low systemic exposure to TCA afforded by the extended release formulation of TCA/PLGA microparticles.

In conclusion, this result suggests that TCA/PLGA microparticles may be administered with minimal disruption of glycemic control in patients with type 2 DM.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. Applicants reserve the right to pursue such inventions in later claims.

What is claimed is:

1. A method of treating pain or inflammation in a patient with diabetes comprising administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40, wherein the formulation is administered at a therapeutically effective concentration that is accompanied by clinically insignificant or no measurable effect on blood glucose and/or does not produce a significant elevation in blood glucose concentration in said diabetic patient following administration.

2. The method of claim 1, wherein the formulation releases TCA for at least 14 days at a rate that does not adversely suppress the hypothalamic-pituitary-adrenal axis (HPA axis).

3. A method of slowing, arresting or reversing progressive structural tissue damage associated with chronic inflammatory disease in a patient with diabetes comprising administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40, wherein the formulation is administered at a therapeutically effective concentration that is accompanied by clinically insignificant or no measurable effect on blood glucose and/or does not produce a significant elevation in blood glucose concentration in said diabetic patient following administration.

4. The method of claim 3, wherein the formulation releases TCA for at least 14 days at a rate that does not adversely suppress the hypothalamic-pituitary-adrenal axis (HPA axis).

5. A method of slowing, arresting, reversing loss of or improving joint function in a patient with diabetes comprising administering to said patient a therapeutically effective amount of a formulation comprising controlled- or sustained-release microparticles comprising triamcinolone acetonide (TCA) or a pharmaceutically-acceptable salt thereof and a poly(lactic-co-glycolic) acid copolymer (PLGA) matrix, wherein the TCA comprises between 22% to 28% of the microparticles and wherein the PLGA has the following characteristics: (i) a molecular weight in the range of about 40 to 70 kDa; and (ii) a lactic acid:glycolic acid molar ratio of 80:20 to 60:40, wherein the formulation is administered at a therapeutically effective concentration that is accompanied by clinically insignificant or no measurable effect on blood glucose and/or does not produce a significant elevation in blood glucose concentration in said diabetic patient following administration.

6. The method of claim 1, wherein the PLGA copolymer has a molar ratio of lactic acid:glycolic acid of 75:25.

7. The method of claim 1, wherein the 22% to 28% of TCA in the microparticles comprises a total TCA load dose between 10 to 50 mg.

8. The method of claim 1, wherein the formulation comprises a TCA dose in of about 40 mg.

9. The method of claim 1, wherein the PLGA has an inherent viscosity in the range of 0.3 to 0.5 dL/g.

10. The method of claim 1, wherein the TCA is released for between 14 days and 90 days.

11. The method of claim 1, wherein the formulation is administered as one or more injections.

12. The method of claim 11, wherein the injection is one or more local injections at a site of pain.

13. The method of claim 11, wherein the injection is one or more intra-articular or periarticular injections.

14. The method of claim 1, wherein the patient has osteoarthritis, rheumatoid arthritis, acute gouty arthritis, and/or synovitis.

15. The method of claim 1, wherein the blood glucose concentration in the subject following intra-articular administration is elevated to a level that is no more than five-fold greater than an upper limit of a control blood glucose level.

16. The method of claim 15, wherein the control blood glucose level is in the range of about 70 to 99 mg/dL for a normal fasting blood glucose concentration or is less than 140 mg/dL for a normal blood glucose concentration two hours after eating.

17. The method of claim 1, wherein the blood glucose concentration in the subject following intra-articular administration is elevated to a level that is in a range between twofold and five-fold greater than an upper limit of a control blood glucose level.

18. The method of claim 17, wherein the control blood glucose level is in the range of about 70 to 99 mg/dL for a normal fasting blood glucose concentration or is less than 140 mg/dL for a normal blood glucose concentration two hours after eating.

19. The method of claim 1, wherein the blood glucose concentration in the subject following intra-articular administration is elevated to a level that is no more than 600 mg/dL.

20. The method of claim 1, wherein the blood glucose concentration in the subject following intra-articular administration is elevated to a level that is no more than 500 mg/dL.

21. The method of claim 1, wherein the blood glucose concentration in the subject following intra-articular administration is elevated to a level that is no more than 400 mg/dL.

22. The method of claim 1, wherein the blood glucose concentration in the subject following intra-articular administration is elevated to a level that is no more than 300 mg/dL.

23. The method of claim 1, wherein the blood glucose concentration in the subject following intra-articular administration is elevated to a level that is no more than 200 mg/dL.

24. The method of claim 1, wherein the blood glucose concentration in the subject following intra-articular administration is elevated to a level that is no more than 100 mg/dL.

25. The method of claim 1, wherein the subject has type 2 diabetes.

26. The method of claim 1, wherein the subject has type 1 diabetes.
